(19) United States
(12) Patent Application Publication
(10) Pub. No.: US 2022/0050090 A1
(43) Pub. Date: Feb. 17, 2022

(54) MASS SPECTROMETRY ASSAY METHOD FOR DETECTION AND QUANTITATION OF MICROBIOTA RELATED METABOLITES

(71) Applicant: Metabolon, Inc., Morrisville, NC (US)

(72) Inventors: Klaus Peter ADAM, Cary, NC (US); Haibao WAN, Cary, NC (US); Gregory M. SCHAAF, Cary, NC (US); Qibo ZHANG, Cary, NC (US)

(21) Appl. No.: 17/274,568

(22) PCT Filed: Sep. 4, 2019
(86) PCT No.: PCT/US19/49422

§ 371 Date: Mar. 9, 2021

Related U.S. Application Data
(60) Provisional application No. 62/823,986, filed on Mar. 26, 2019.
(60) Provisional application No. 62/767,552, filed on Nov. 15, 2018.
(60) Provisional application No. 62/729,444, filed on Sep. 11, 2018.

(51) Int. Cl. G01N 30/72; G01N 33/68

(52) U.S. Cl. CPC G01N 30/724; G01N 2458/15; G01N 33/6848

(57) ABSTRACT

A method for determining in a sample, by mass spectrometry, the amount of one or more analytes is described. The method comprises introducing a sample to an ionization source under conditions suitable to produce one or more ions detectable by mass spectrometry from each of the one or more analytes; measuring, by mass spectrometry, the amount of the one or more ions from each of the one or more analytes and using the measured amount of the one or more ions to determine the amount of each of the one or more analytes in the sample. Also described is a kit comprising one or more isotopically labeled analogues as internal standards for each of the one or more analytes.

### BACKGROUND

The following information to describe the background of the invention is provided to assist the understanding of the invention and is not admitted to constitute or describe prior art to the invention.

Alterations in microflora (i.e., dysbiosis, including changes to the microbiome composition, or a microbial imbalance on or inside the body) and its activities have been associated with, and are now believed to be contributing factors to, many chronic and degenerative diseases such as allergies, arthritis, asthma, autism, colon cancer, C. difficile infections, diabetes, IBS, obesity and others.

Recent advances in DNA-based technologies, have enabled the exploration of the diversity and abundance in the human microbiome. Efforts are underway to use these technologies to characterize the genes present in all microorganisms that permanently live in different sites of the human body. This work is aimed at unravelling bacterial gene functions and their role in human health.

Microbial communities rely on small molecules for communication within and across species, including animal species (hosts). There is increasing evidence that these communications between host and microbe impact human health, and the health impact may be beneficial or detrimental. However, the microbe-host interactions underlying these effects are not well-understood. The ability to measure changes in the levels of microbial and microbial-associated analytes could provide an indication of mechanistic details of microbiota-induced changes in a host and the resulting health effects. In addition, insight into the health of the microbiome itself may be obtained. Therefore, a method to detect and measure the levels of microbial and microbial-associated analytes could provide insight into what constitutes a healthy microbiome and represents a significant unmet medical need.

Described herein are methods for the detection and quantitation of one or more of, a plurality of, or a panel of, analytes useful for the assessment of the microbiota and host-microbe interactions in a subject. The results of these methods allow for quantitative measurement of a variety of structurally diverse microbial and microbiome-related analytes in a small volume sample. The metabolite assays can be performed using mass spectrometry analysis methods, require only a single sample injection per assay or panel and do not require derivatization.

### SUMMARY

In a first aspect of the invention, a method comprises detecting and determining the amount of a panel of analytes comprised of one or a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate (3-(1H-Imidazol-4-yl)propionic acid, Deamino-histidine), imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid (indolelactate), and combinations thereof in a sample by mass spectrometry. In one embodiment, the method comprises subjecting the sample to an ionization source under conditions suitable to produce one or more ions detectable by mass spectrometry from each of the one or more analytes. In another embodiment, the analytes are not derivatized prior to ionization. Methods to extract the analytes from samples, and to chromatographically separate the analytes prior to detection by mass spectrometry are also provided.

In a second aspect of the invention, a method for determining the amount of one or a plurality of analytes in a sample by mass spectrometry is described. The one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof. The steps include introducing the sample to an ionization source under conditions suitable to produce one or more ions detectable by mass spectrometry from each of the one or plurality of analytes, wherein the analytes are not derivatized prior to ionization; measuring, by mass spectrometry, the amount of the one or more ions from each of the one or plurality of analytes; and using the measured amount of the one or more ions to determine the amount of each of the one or plurality of analytes in the sample. In one feature of the aspect, the mass spectrometry is tandem mass spectrometry. In another feature of the aspect, the one or more ions used to determine the amount of each of the plurality analytes are one or more ions selected from the ions in Tables 3, 4, and 5. In a further feature of the aspect, when the one of the one or plurality of analytes comprises N-palmitoyl serinol, the one or more ions comprise one or more ions selected from the group consisting of ions with a mass to charge ratio of 330.3±0.5, 312.1±0.5, 239.1±0.5, 149.1±0.5, 139.1±0.5, 92.1±0.5, and 74.1±0.5.

In a feature of the first and second aspect, the one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and the amounts of the one or plurality of analytes are determined in a single injection. With respect to this feature, the ionization source is operated in positive ionization mode.

In another feature, the one or plurality of analytes are selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and the amounts of the one or plurality of analytes are determined in a single injection. With respect to this feature, the ionization source is operated in negative ionization mode.

In another feature, the one or plurality of analytes are selected from the group consisting of xylose, raffinose, stachyose, N-acetylmuraminate, and N-acetylneuraminate (sialic acid), and the amount(s) of the one or plurality of analytes are determined in a single injection. With respect to this feature, the ionization source is operated in negative ionization mode.

In another feature, the one or plurality of analytes are selected from the group consisting of catechol sulfate, p-cresol sulfate, ethylphenyl sulfate, indole lactate, indolepropionate, and indoxyl sulfate, and the amount(s) of the one or plurality of analytes are determined in a single injection. With respect to this feature, the ionization source is operated in negative ionization mode.

In another feature, the analyte is trimethylamine-N-oxide (TMAO), and the amount of the analyte is determined in a single injection. With respect to this feature, the ionization source is operated in positive ionization mode.

In another feature, the sample has been purified by liquid chromatography prior to being introduced to the ionization source. With respect to this feature, the liquid chromatography is selected from the group consisting of high performance liquid chromatography, ultra high performance liquid chromatography, and turbulent flow liquid chromatography. With further regard to this feature, the sample is purified by either high performance liquid chromatography or ultrahigh performance liquid chromatography prior to being introduced to the ionization source.

In further features, the amount of two or more, three or more, four or more, five or more, six or more, or seven or more of the plurality of analytes are determined.

In another feature, the amount of N-palmitoyl serinol and one or more analytes selected from the group consisting of indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, and 3-indolelactic acid is determined.

In yet another feature, a first one or more analyte(s) of the plurality of analytes is selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and the first one or more analyte(s) of the plurality of analytes are determined in a single injection; and a second one or more analyte(s) of the plurality of analytes is selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and the second one or more analyte(s) of the plurality of analytes are determined in a single injection. With regard to this feature, a third one or more analyte(s) of the plurality of analytes is selected from the group consisting of xylose, raffinose, stachyose, N-acetylmuraminate, and N-acetylneuraminate (sialic acid), and the third one or more analyte(s) of the plurality of analytes are determined in a single injection.

In another feature, one or more internal standards are used to determine the amount of each of the one or plurality of analytes in the sample. With regard to this feature, at least one of the one or more internal standards comprises an isotopically labeled analog of at least one of the one or plurality of analytes to be measured. With further regard to this feature, the at least one of the one or more internal standards are selected from the group consisting of N-palmitoyl serinol-d₃, trimethylamine N-oxide-¹³C₃, 3-indolepropionic acid-d₂, indole-d₇, N-acetylcadaverine-d₃, 5-aminovaleric acid-d₄, cadaverine-d₄, famotidine-¹³C₃, gamma-aminobutyric acid-d₆, serotonin-d₄, pipecolic acid-d₉, imidazole propionic acid-d₃, imidazolelactic acid-d₃, cylco(-His-Pro)-d₃, cyclo(-Pro-Thr)-d₃, cyclo(-Gly-His)-d₄, tryptophan-d₅, p-cresol-d₇, benzoic acid-d₅, hippurate-d₅, 4-hydroxyphenylacetic acid-d₆, 3-phenyllactic acid-d₅, (4-hydroxyphenyl)-2-propionic acid-d₆, naringenin-d₃, (3-phenylpropionyl)glycine-¹³C₂, ¹⁵N₁, phenylacetylglycine-d₅, p-cresol sulfate-d₇, enterodiol-d₆, enterolactone-d₆, phenol sulfate-d₃, daidzein-d₄, apigenin-d₅, p-cresol glucuronide-d₇, genistein-d₄, ethylphenyl sulfate-d₄, equol-d₄, 3-indoxyl sulfate-¹³C₆, phenylacetic acid-d₇, deoxycholic acid-d₄, lithocholic acid-d₄, taurodeoxycholic acid-d₅, lactic acid-d₄, xylose-¹³C₅, raffinose-d₉, stachyose-d₇, diaminopimelic acid-¹³C₇, ¹⁵N₂, trimethylamine-¹³C₃, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, urolithin B-¹³C₆, N-acetylmuramic acid-d₃, N-acetyl-D-neuraminic acid-1,2,3-¹³C₃, catechol sulfate-¹³C₆, and indolelactate-d₅.

In a third aspect of the invention, a kit comprises one or more isotopically labeled analogs as internal standards for each of one or a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, and 3-indolelactic acid and combinations thereof, and packaging material and instructions for using the kit. With regard to this feature, the internal standards comprise one or more internal standards selected from the group consisting of N-palmitoyl serinol-d₃, trimethylamine N-oxide-¹³C₃, 3-indolepropionic acid-d₂, indole-d₇, N-acetylcadaverine-d₃, 5-aminovaleric acid-d₄, cadaverine-d₄, famotidine-¹³C₃, gamma-aminobutyric acid-d₆, serotonin-d₄, pipecolic acid-d₉, imidazole propionic acid-d₃, imidazolelactic acid-d₃, cylco(-His-Pro)-d₃, cyclo(-Pro-Thr)-d₃, cyclo(-Gly-His)-d₄, tryptophan-d₅, p-cresol-d₇, benzoic acid-d₅, hippurate-d₅, 4-hydroxyphenylacetic acid-d₆, 3-phenyllactic acid-d₅, (4-hydroxyphenyl)-2-propionic acid-d₆, naringenin-d₃, (3-phenylpropionyl)glycine-¹³C₂, ¹⁵N₁, phenylacetylglycine-d₅, p-cresol sulfate-d₇, enterodiol-d₆, enterolactone-d₆, phenol sulfate-d₃, daidzein-d₄, apigenin-d₅, p-cresol glucuronide-d₇, genistein-d₄, ethylphenyl sulfate-d₄, equol-d₄, 3-indoxyl sulfate-¹³C₆, phenylacetic acid-d₇, deoxycholic acid-d₄, lithocholic acid-d₄, taurodeoxycholic acid-d₅, lactic acid-d₄, xylose-¹³C₅, raffinose-d₉, stachyose-d₇, diaminopimelic acid-¹³C_{7,}¹⁵N₂, trimethylamine-¹³C₃, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, urolithin B-¹³C₆, N-acetylmuramic acid-d₃, N-acetyl-D-neuraminic acid-1,2,3-¹³C₃, and combinations thereof.

In a feature of the third aspect, the one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and combinations thereof. With regard to this feature, the internal standards comprise one or more internal standards selected from the group consisting of N-palmitoyl serinol-d₃, 3-indolepropionic acid-d₂, indole-d₇, tryptophan-d₅, 5-aminovaleric acid-d₄, pipecolic acid-d₉, N-acetylcadaverine-d₃, cadaverine-d₄, trimethylamine N-oxide-¹³C₃, gamma-aminobutyric acid-d₆, serotonin-d₄, imidazole propionic acid-d₃, imidazolelactic acid-d₃, cylco(-His-Pro)-d₃, cyclo(-Pro-Thr)-d₃, cyclo(-Gly-His)-d₄, famotidine-¹³C₃, diaminopimelic acid-¹³C_{7,}¹⁵N₂, trimethylamine-¹³C₃, catechol sulfate-¹³C₆, indolelactate-d₅, and combinations thereof.

In another feature, the one or plurality of analytes are selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and combinations thereof. With regard to this feature, the internal standards comprise one or more internal standards selected from the group consisting of p-cresol-d₇, 3-indoxyl sulfate-¹³C₆, 4-hydroxyphenylacetic acid-d₆, (4-hydroxyphenyl)-2-propionic acid-d₆, benzoic acid-d₅, phenylacetic acid-d₇, 3-phenyllactic acid-d₅, hippurate-d₅, lactic acid-d₄, (3-phenylpropionyl)glycine-¹³C_{2,}¹⁵N₁, phenylacetylglycine-d₅, ethylphenyl sulfate-d₄, phenol sulfate-d₃, p-cresol sulfate-d₇, p-cresol glucuronide-d₇, enterodiol-d₆, enterolactone-d₆, equol-d₄, daidzein-d₄, apigenin-d₅, naringenin-d₃, genistein-d₄, deoxycholic acid-d₄, lithocholic acid-d₄, taurodeoxycholic acid-d₅, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, urolithin B-¹³C₆, and combinations thereof.

In yet another feature, the one or plurality of analytes are selected from the group consisting of xylose, raffinose, stachyose, N-acetylmuraminate, and N-acetylneuraminate (sialic acid), and combinations thereof. With regard to this feature, the internal standards comprise one or more internal standards selected from the group consisting of xylose-¹³C₅, raffinose-d₉, stachyose-d₇, N-acetylmuramic acid-d₃, N-acetyl-D-neuraminic acid-1,2,3-¹³C₃, and combinations thereof.

In yet another feature, the one or plurality of analytes are selected from the group consisting of catechol sulfate, p-cresol sulfate, ethylphenyl sulfate, indole lactate, indolepropionate, indoxyl sulfate, and combinations thereof. With regard to this feature, the internal standards comprise one or more internal standards selected from the group consisting of catechol sulfate-¹³C₆, p-cresol sulfate-d₇, ethylphenyl sulfate-d₄, indolelactate-d₅, indolepropionate-d₂, 3-Indoxyl sulfate-¹³C₆, and combinations thereof.

In yet another feature, the analyte is trimethylamine-N-oxide (TMAO). With regard to this feature, the internal standard comprises trimethylamine N-oxide-¹³C₃.

In another feature of the third aspect, the one or plurality analytes are selected from the group consisting of N-acetyl-cadaverine, 5-aminovalerate, imidazole propionate, β-imidazolelactic acid, N-palmitoyl serinol, cylco(-His-Pro), cyclo(-Pro-Thr), cyclo(-Gly-His), 2-(4-hydroxyphenyl)propionate, naringenin, phenol sulfate, ethylphenyl sulfate, raffinose, stachyose, 4-hydroxyphenyllactate, phenol glucuronide, N-acetylmuraminate, catechol sulfate, and combinations thereof. With regard to this feature, the one or more internal standards are selected from the group consisting of N-acetyl-cadaverine-d₃, 5-aminovalerate-d₄, imidazole propionate-d₃, β-imidazolelactic acid-d₃, N-palmitoyl serinol-d₃, cylco(-His-Pro)-d₃, cyclo(-Pro-Thr)-d₃, cyclo(-Gly-His)-d₄, 2-(4-hydroxyphenyl)propionate-d₆, naringenin-d₃ sodium salt, phenol sulfate-d₃, ethylphenyl sulfate-d₄, raffinose-d₉, stachyose-d₇, 4-hydroxyphenyllactate-d₂, phenol glucuronide-d₅, N-acetylmuramic acid-d₃, catechol sulfate-¹³C₆, and combinations thereof.

### BRIEF DESCRIPTION OF THE DRAWINGS

FIGS. 1A-B show example chromatograms of N-acetyl-cadaverine, imidazole propionate, pipecolate, indole, 5-aminovalerate, gamma-aminobutyric acid (GABA), cadaverine, trimethylamine-N-oxide (TMAO), famotidine, N-palmitoyl serinol, cyclo(-His-Pro), tryptophan, cyclo(-Pro-Thr), cyclo(-Gly-His), indolepropionate, serotonin, and imidazole lactate, purified and separated in a single injection using Chromatography Method 1.

FIGS. 2A-C show example chromatograms of phenol sulfate, phenyllactate, 2-(4-hydroxyphenyl)propionate, 4-hydroxyphenylacetate, phenylacetic acid, benzoate, cresol, lactate, daidzein, equol, 3-indoxyl sulfate, phenylpropionylglycine, ethylphenyl sulfate, phenylacetylglycine, p-cresol sulfate, hippurate, taurodeoxycholate, deoxycholate, lithocholate, enterodiol, enterolactone, p-cresol glucuronide, naringenin, genistein, and apigenin, purified and separated in a single injection using Chromatography Method 2.

FIG. 3 shows example chromatograms of xylose, raffinose, and stachyose, purified and separated in a single injection using Chromatography Method 3.

FIG. 4 shows example chromatograms of catechol sulfate, p-cresol sulfate, ethylphenyl sulfate, indole lactate, indolepropionate, and indoxyl sulfate purified and separated in a single injection using Chromatography Method 4.

FIG. 5 shows an exemplary chromatogram of trimethylamine-N-oxide (TMAO) purified and separated using Chromatography Method 5.

### DETAILED DESCRIPTION

Methods are described for measuring the amount of one or more analytes or a plurality of analytes selected from the group of metabolites consisting of: N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof, in a sample. Mass spectrometric methods are described for quantifying single and multiple (a plurality) analytes in a sample using a single injection method. In examples where a plurality of analytes is quantified, the analytes may be referred to as a “panel” or a “panel of analytes”. In one example, the panel may comprise a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof. In another example, the panel may comprise a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, trimethylamine (TMA), and combinations thereof. In yet another example, the panel may comprise a plurality of analytes selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, urolithin A, and combinations thereof. In yet a further example, the panel may comprise a plurality of analytes selected from the group consisting of xylose, raffinose, stachyose, N-acetylmuraminate, N-acetylneuraminate (sialic acid), and combinations thereof. In another example, the panel may comprise a plurality of analytes selected from the group consisting of catechol sulfate, p-cresol sulfate, ethylphenyl sulfate, indole lactate, indolepropionate, indoxyl sulfate, and combinations thereof. The methods may include a purification or enrichment step using, for example, a liquid chromatography step such as LC (liquid chromatography), UPLC (ultra-high performance liquid chromatography) or HILIC (hydrophilic interaction chromatography) to perform a separation (purification, enrichment) of selected analytes combined with methods of mass spectrometry. An advantage of the methods described herein is the provision of a high-throughput assay system that is amenable to automation for quantifying a plurality of analytes in a sample.

The methods presented herein provide improvements and advantages over current methods to measure these analytes. Methods for measuring multiple panels of analytes are provided. The analytes included in the panels are structurally diverse, and the methods provide a technical improvement and advantage by measuring the analytes together in a single injection without derivatization. Further, in this method, a stable isotope-labeled analog of the analyte is used for each individual analyte as an internal standard. Using a labeled analog for each analyte allows for more accurate quantitation than methods that use one internal standard to quantitate several (e.g., 3 or more) analytes or use a structurally similar labeled compound (but not an analog) for quantitation. The ability to quantifiably measure, in a single injection, a plurality of analytes in various combinations, reduces the time required to obtain analysis results, uses fewer resources in terms of laboratory disposables (e.g., tubes, pipette tips, reagents), laboratory instruments and human resources. These improvements lead to savings by decreasing the costs of the assays and increasing the instrument and laboratory capacity for sample analysis.

Prior to describing this invention in further detail, the following terms are defined.

### Definitions

The term “amount” means the quantity of the analyte that is measured using the methods described herein. The amount may be expressed as a concentration. For example, mass concentration, molar concentration, number concentration, or volume concentration. Amount as used herein refers to an absolute amount or absolute quantity as opposed to a relative amount or relative quantity.

The term “solid phase extraction” refers to a sample preparation process where components of complex mixture (i.e., mobile phase) are separated according to their physical and chemical properties using solid particle chromatographic packing material (i.e. solid phase or stationary phase). The solid particle packing material may be contained in a cartridge type device (e.g. a column).

The term “separation” refers to the process of separating a complex mixture into its component molecules or metabolites. Common, exemplary laboratory separation techniques include electrophoresis and chromatography.

The term “chromatography” refers to a physical method of separation in which the components (i.e., chemical constituents) to be separated are distributed between two phases, one of which is stationary (stationary phase) while the other (the mobile phase) moves in a definite direction. The mobile phase may be gas (“gas chromatography”, “GC”) or liquid (“liquid chromatography”, “LC”). Chromatographic output data may be used in embodiments of the method described herein.

The term “liquid chromatography” or “LC” refers to a process of selective inhibition of one or more components of a fluid solution as the fluid uniformly moves through a column of a finely divided substance or through capillary passageways. The inhibition results from the distribution of the components of the mixture between one or more stationary phases and the mobile phase(s) as the mobile phase(s) move relative to the stationary phase(s). Examples of “liquid chromatography” include “Reverse phase liquid chromatography” or “RPLC”, “high performance liquid chromatography” or “HPLC”, “ultra-high performance liquid chromatography” or “UPLC” or “UHPLC”, or hydrophilic interaction chromatography or “HILIC”.

The term “retention time” refers to the elapsed time in a chromatography process since the introduction of the sample into the separation device. The retention time of a constituent of a sample refers to the elapsed time in a chromatography process between the time of injection of the sample into the separation device and the time that the constituent of the sample elutes (e.g., exits from) the portion of the separation device that contains the stationary phase.

The term “retention index” of a sample component refers to a number, obtained by interpolation (usually logarithmic), relating the retention time or the retention factor of the sample component to the retention times of standards eluted before and after the peak of the sample component, a mechanism that uses the separation characteristics of known standards to remove systematic error.

The term “separation index” refers to a metric associated with chemical constituents separated by a separation technique. For chromatographic separation techniques, the separation index may be retention time or retention index. For non-chromatographic separation techniques, the separation index may be physical distance traveled by the chemical constituent.

As used herein, the terms “separation information” and “separation data” refer to data that indicates the presence or absence of chemical constituents with respect to the separation index. For example, separation data may indicate the presence of a chemical constituent having a particular mass eluting at a particular time. The separation data may indicate that the amount of the chemical constituent eluting over time rises, peaks, and then falls. A graph of the presence of the chemical constituent plotted over the separation index (e.g., time) may display a graphical peak. Thus, within the context of separation data, the terms “peak information” and “peak data” are synonymous with the terms “separation information” and “separation data”.

The term “Mass Spectrometry” (MS) refers to a technique for measuring and analyzing molecules that involves ionizing or ionizing and fragmenting a target molecule, then analyzing the ions, based on their mass/charge ratios, to produce a mass spectrum that serves as a “molecular fingerprint”. Determining the mass/charge ratio of an object may be done through means of determining the wavelengths at which electromagnetic energy is absorbed by that object. There are several commonly used methods to determine the mass to charge ratio of an ion, some measuring the interaction of the ion trajectory with electromagnetic waves, others measuring the time an ion takes to travel a given distance, or a combination of both. The data from these fragment mass measurements can be searched against databases to obtain identifications of target molecules.

The terms “operating in negative mode” or “operating in negative multiple reaction monitoring (MRM) mode” or “operating in negative ionization mode” refer to those mass spectrometry methods where negative ions are generated and detected. The terms “operating in positive mode” or “operating in positive multiple reaction monitoring (MRM) mode” or “operating in positive ionization mode” refer to those mass spectrometry methods where positive ions are generated and detected.

The term “mass analyzer” refers to a device in a mass spectrometer that separates a mixture of ions by their mass-to-charge (“m/z”) ratios.

The term “m/z” refers to the dimensionless quantity formed by dividing the mass number of an ion by its charge number. It has long been called the “mass-to-charge” ratio.

As used herein, the term “source” or “ionization source” refers to a device in a mass spectrometer that ionizes a sample to be analyzed. Examples of ionization sources include electrospray ionization (ESI), atmospheric pressure chemical ionization (APCI), heated electrospray ionization (HESI), atmospheric pressure photoionization (APPI), flame ionization detector (FID), matrix-assisted laser desorption ionization (MALDI), etc.

As used herein, the term “detector” refers to a device in a mass spectrometer that detects ions.

The term “ion” refers to any object containing a charge, which can be formed for example by adding electrons to or removing electrons from the object.

The term “mass spectrum” refers to a plot of data produced by a mass spectrometer, typically containing m/z values on x-axis and intensity values on y-axis.

The term “scan” refers to a mass spectrum that is associated with a particular separation index. For example, systems that use a chromatographic separation technique may generate multiple scans, each scan at a different retention time.

The term “run time”, refers to the time from sample injection to generation of the instrument data.

The term “tandem MS” refers to an operation in which a first MS step, called the “primary MS”, is performed, followed by performance of one or more of a subsequent MS step, generically referred to as “secondary MS”. In the primary MS, an ion, representing one (and possibly more than one) chemical constituent, is detected and recorded during the creation of the primary mass spectrum. The substance represented by the ion is subjected to a secondary MS, in which the substance of interest undergoes fragmentation in order to cause the substance to break into sub-components, which are detected and recorded as a secondary mass spectrum. In a true tandem MS, there is an unambiguous relationship between the ion of interest in the primary MS and the resulting peaks created during the secondary MS. The ion of interest in the primary MS corresponds to a “parent” or precursor ion, while the ions created during the secondary MS correspond to sub-components of the parent ion and are herein referred to as “daughter” or “product” ions.

Thus, tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion. Tandem MS may be repeated on daughter ions to determine “grand-daughter” ions, for example. Thus, tandem MS is not limited to two-levels of fragmentation, but is used generically to refer to multi-level MS, also referred to as “MSⁿ”. The term “MS/MS” is a synonym for “MS²”. For simplicity, the term “daughter ion” hereinafter refers to any ion created by a secondary or higher-order (i.e., not the primary) MS.

“Analyte”, “small molecule”, “biochemical” or, “metabolite” may be used interchangeably. As used herein, examples of analytes include N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, and N-acetylneuraminate (sialic acid). The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000).

“Sample” refers to the material to be analyzed by the described methods. Samples may be solid samples, liquid samples or volatile samples. Sample may refer to any type of sample and may include non-biological samples (non-limiting examples include: soil samples, water samples, solid formulations, (including but limited to, for example, food samples) liquid formulations (including but not limited to, for example, beverage samples), and biological samples. The term “biological sample”, may refer to biological material isolated from a subject. A biological sample may contain any biological material suitable for detecting the desired analyte(s), and may comprise cellular and/or non-cellular material from a subject. Non-limiting examples of biological samples include: blood, blood plasma (plasma), blood serum (serum), urine, cerebral spinal fluid (CSF), feces, tissue, skin, cecal content, breast milk, saliva, plant samples, cells or cell cultures, cell culture medium, and biofilms.

“Subject” means any animal, but is preferably a mammal, such as, for example, a human, monkey, mouse, dog, rabbit or rat.

### I. Sample Preparation and Quality Control (QC)

Sample extracts containing analytes are prepared by isolating the analytes in the sample from the macromolecules (e.g., proteins, nucleic acids, lipids) that may be present in the sample. The terms “sample extracts”, “extracted samples” or “analyte extracts” may also be referred to herein as “analytical samples” and the terms may be used interchangeably. Some or all analytes in a sample may be bound to proteins. Various methods may be used to disrupt the interaction between analyte(s) and protein prior to MS analysis. For example, the analytes may be extracted from a sample to produce a liquid extract, while the proteins that may be present are precipitated and removed. Proteins may be precipitated using, for example, a solution of ethyl acetate or methanol. To precipitate the proteins in the sample, an ethyl acetate or methanol solution is added to the sample, then the mixture may be spun in a centrifuge to separate the liquid supernatant, which contains the extracted analytes, from the precipitated proteins

In other embodiments, analytes may be released from protein without precipitating the protein. For example, a formic acid solution may be added to the sample to disrupt the interaction between protein and analyte. Alternatively, ammonium sulfate, a solution of formic acid in ethanol, or a solution of formic acid in methanol may be added to the sample to disrupt ionic interactions between protein and analyte without precipitating the protein. In one example, a solution of acetonitrile, methanol, water, and formic acid may be used to extract analytes from the sample.

In some embodiments the extract may be subjected to various methods including liquid chromatography, electrophoresis, filtration, centrifugation, and affinity separation as described herein to purify or enrich the amount of the selected analyte relative to one or more other components in the sample.

To assess, for example, precision, accuracy, calibration range, or analytical sensitivity of methods of detecting and quantifying analytes, quality control (QC) samples may be used. The concentration of a given analyte(s) to be used in a QC sample may be determined based on lower limit of quantitation (LLOQ) or upper limit of quantitation (ULOQ) of the given analyte(s), as detected in a sample. In one example, the LLOQ may be represented by the concentration of a standard (e.g., Standard A), and the ULOQ may be represented by the concentration of a second standard (e.g., Standard H). The Low QC value may be set at a concentration of about 3× LLOQ, the Mid QC value may be at a concentration of about 25-50% of High QC, and the High QC value may be at a concentration of about 80% of the ULOQ. The QC target concentration levels may be chosen based on a combination of the Analytical Measurement Range (AMR) and the frequency of sample results as measured in a set of representative samples.

### II. Chromatography

Prior to mass spectrometry, the analyte extract may be subjected to one or more separation methods such as electrophoresis, filtration, centrifugation, affinity separation, or chromatography. In one embodiment the separation method may comprise liquid chromatography (LC), including, for example, ultra high performance LC (UHPLC).

In some embodiments, UHPLC may be conducted using a reversed phase column chromatographic system, hydrophilic interaction chromatography (HILIC), or a mixed phase column chromatographic system.

The column heater (or column manager) for LC may be set at a temperature of from about 25° C. to about 80° C. For example, the column heater may be set at about 30° C., 40° C., 50° C., 60° C., 70° C., etc.

In an example, UHPLC may be conducted using a HILIC system. In another example, UHPLC may be conducted using a reversed phase column chromatographic system. The system may comprise two or more mobile phases. Mobile phases may be referred to as, for example, mobile phase A, mobile phase B, mobile phase A′, and mobile phase B′.

In an exemplary embodiment using two mobile phases, A and B, mobile phase A may comprise perfluoropentanoic acid (PFPA) and water, and mobile phase B may comprise PFPA and acetonitrile. The concentration of PFPA may be from about 0.01 to about 0.500%. The concentration of acetonitrile may range from 0% to 100%. In some examples, the concentration of perfluoropentanoic acid (PFPA) in mobile phase A may be 0.03, 0.04, 0.05, 0.06, 0.07, 0.08, 0.09, 0.1, 0.2, or 0.3%. In other examples, the concentration of PFPA in mobile phase B may be 0.03, 0.04, 0.05, 0.06, 0.07, 0.08, 0.09, 0.1, 0.2, or 0.3%, and the concentration acetonitrile may be 99.97, 99.96, 99.95, 99.94, 99.93, 99.92, 99.91, 99.9, 99.8, or 99.7%.

In one example, linear gradient elution may be used for chromatography. The starting conditions for linear gradient elution may include the concentration of a mobile phase (e.g., mobile phase B) and/or the flow rate of a mobile phase through the column (e.g., mobile phase B). The starting conditions may be optimized for the separation and/or retention of one or more analytes. The gradient conditions may also be optimized for the separation and/or retention of analytes and may vary depending on the flow rate selected. For example, initial conditions may be 0.5% mobile phase B and 600 μL/min flow rate. Mobile phase B may be increased to 5-10% by about 4 minutes, increased to about 40-90% at about 5.5-6.0 minutes, and increased to about 90-98% at about 6.5 min. Mobile phase B may revert to 0.5% at 6.7 min where it may be maintained for less than a minute for equilibration for the next sample injection. The total run time may be 7.0 minutes or less.

In another example, mobile phase A may comprise formic acid and water, and mobile phase B may comprise formic acid and acetonitrile. The concentration of formic acid in mobile phase A or mobile phase B may range from 0.001% to 5%. The concentration of acetonitrile may range from 0% to 100%. For example, mobile phase A may comprise 0.005, 0.01, 0.05, 0.1, or 0.5% formic acid in water and mobile phase B may comprise 0.005, 0.01, 0.05, 0.1, or 0.5% formic acid in acetonitrile. Linear gradient elution may be used for chromatography and may be carried out with an initial condition of 0% mobile phase B and a flow rate of 600 μL/min. Mobile phase B may then be increased to 20-25% at 3.5-4 min, increased to 25-30% at 6.5-6.9 min, increased to 70-90% at 6.9-8.0 min, increased to 90-95% at 8-8.5 min. Mobile phase B may then be maintained at 95% for less than 0.5 min. Mobile phase B may revert to 0% for less than a minute for equilibration before the next sample injection. The total run time may be 9.0 minutes or less.

In yet another example, mobile phase A may comprise triethylamine and water, and mobile phase B may comprise triethylamine and acetonitrile. The concentration of triethylamine may range from about 0.01 to about 0.500%, and the concentration of acetonitrile may range from 0% to 100%. In some examples, the concentration of triethylamine in mobile phase A or mobile phase B may be 0.005, 0.01, 0.05, 0.1, or 0.5%. Linear gradient elution may be used for chromatography. For example, initial conditions may be 2% mobile phase A and 600 μL/min flow rate. Mobile phase A may be increased to about 10-20% at 1.5-2.0 minutes, increased to 25-30% at about 5 minutes, increased to 40-50% at about 5 minutes and maintained for less than 0.5 min. Mobile phase A may revert to 2% at about 5.5 min where it may be maintained for about 0.5 min for equilibration for the next sample injection. The total run time may be 6.0 minutes or less.

In a further example, mobile phase A may comprise formic acid and water, and mobile phase B may comprise formic acid and acetonitrile. The concentration of formic acid in mobile phase A or mobile phase B may range from 0.001% to 5%. The concentration of acetonitrile may range from 0% to 100%. For example, mobile phase A may comprise 0.005, 0.01, 0.05, 0.1, or 0.5% formic acid in water and mobile phase B may comprise 0.005, 0.01, 0.05, 0.1, or 0.5% formic acid in acetonitrile. Linear gradient elution may be used for chromatography and may be carried out with an initial condition of 0-15% mobile phase B and a flow rate of 550 μL/min. Mobile phase B may then be increased to 15-30% at about 3 min, increased to 30-45% at 4.0-4.3 min, and increased to 70-99% at 4.3-5.0 min. Mobile phase B may revert to 10% for less than a minute for equilibration before the next sample injection. The total run time may be 5.50 minutes or less.

In yet a further example, mobile phase A may comprise ammonium formate and water, and mobile phase B may comprise ammonium formate, acetonitrile, and water. The concentration of ammonium formate in mobile phase A may range from 0.1 mM to 100 mM, and the concentration of acetonitrile may range from 0% to 100%. In some examples, the concentration of ammonium formate in mobile phase A may be 1 mM, 5 mM, 10 mM, 15 mM, 20 mM, 25 mM, or 50 mM, and the concentration of acetonitrile may be 60, 70, 80, or 90%. Linear gradient elution may be used for chromatography and may be carried out with an initial condition of 0-15% mobile phase A and a flow rate of 550 μL/min. Mobile phase A may then be increased to 15-35% at about 2.5 min and increased to 30-60% at 2.6-3.5 min. Mobile phase A may then revert to 5% for less than a minute for equilibration before the next sample injection. The total run time may be 4.30 minutes or less.

The eluent from the chromatography column may be directly and automatically introduced into the electrospray source of a mass spectrometer.

### III. Mass Spectrometry and Quantitation

One or more analytes may be ionized by, for example, mass spectrometry. Mass spectrometry is performed using a mass spectrometer that includes an ionization source for ionizing the fractionated sample and creating charged molecules for further analysis. Ionization of the sample may be performed by, for example, electrospray ionization (ESI). Other ionization sources may include, for example, atmospheric pressure chemical ionization (APCI), heated electrospray ionization (HESI), atmospheric pressure photoionization (APPI), flame ionization detector (FID), or matrix-assisted laser desorption ionization (MALDI). The choice of ionization method may be determined based on a number of considerations. Exemplary considerations include the analyte to be measured, type of sample, type of detector, and the choice of positive or negative mode. In some examples, mass spectrometry methods may be divided into two or more periods to increase sensitivity.

The one or more analytes may be ionized in positive or negative mode to create one or more ions. For example, the analytes N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), may be ionized in positive mode. In yet another example, the analytes cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, urolithin A, xylose, raffinose, stachyose, N-acetylmuraminate, N-acetylneuraminate (sialic acid), catechol sulfate, and 3-indolelactic acid may be ionized in negative mode. In some examples, analytes may be ionized in positive mode and negative mode in a single injection.

In one example, the analytes N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), may be ionized in positive mode and may be measured in a single injection. In another example, the analytes cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, may be ionized in negative mode and may be measured in a single injection. In yet another example, the analytes xylose, raffinose, stachyose, N-acetylmuraminate, and N-acetylneuraminate (sialic acid), may be ionized in negative mode and may be measured in a single injection. In yet another example, the analytes catechol sulfate, p-cresol sulfate, ethylphenyl sulfate, indole lactate, indolepropionate, and indoxyl sulfate, may be ionized in negative mode and may be measured in a single injection.

Mass spectrometer instrument settings may be optimized for the given analysis method and/or for the particular mass spectrometer used. The instrument may use various gases, for example, nitrogen, helium, argon, or zero air. In an embodiment, mass spectrometry may be performed using AB Sciex QTrap 6500 mass spectrometers. In one example, the mass spectrometer may be operated in positive multiple reaction monitoring (MRM) mode. The ionspray voltage setting may range from about 0.5 kV to about 6.0 kV; in one embodiment the voltage may be set at 5.5 kV. The source temperature may range from about 350° C. to about 600° C.; in one embodiment the source temperature may be set at 500° C. The curtain gas may range from about 10 to about 55 psi; in one embodiment the curtain gas is set at 35 psi. The nebulizer and desolvation gas flow rates may range from about 0 to about 90 psi. In one embodiment the flow rates may be set at 70. The CAD gas setting may range from high to low; in one embodiment the collisionally activated dissociation (CAD) gas is set at medium. Declustering potential may range from about 20V to about 190V. The collision energy (CE) may range from about 10 V to about 70 V. The entrance potential (EP) may be about 10V. The collision cell exit potential (CXP) setting may range from about 2V to about 30V.

In another example, the MS instrument may be operated in negative MRM mode. Ionspray voltage settings may range from −0.5 kV to −5.5 kV; in one embodiment the voltage may be set at −4.5 kV. In another embodiment, the voltage may be set at −3.5 kV. The source temperature may range from about 350° C. to 600° C.; in one embodiment the source temperature may be set at 500° C. The curtain gas may range from 10 to 40; in an embodiment the curtain gas may be set at 35. In another embodiment, the curtain gas may be set at 20. The nebulizer and desolvation gas flow rates may range from 40 to 90. In one embodiment the flow rates may be set at 70. In another embodiment, the flow rates may be set at 60. The CAD gas may range from low to high. In one example the CAD may be set, for example, at medium. Declustering potential may range from about −10V to about −290V. The collision energy (CE) may range from about −10 V to about −130 V. The entrance potential (EP) may be about −10V. The collision cell exit potential (CXP) setting may range from about −5V to about −35V.

Following ionization, the charged ions may be analyzed to determine a mass-to-charge ratio. Exemplary suitable analyzers for determining mass-to-charge ratios include quadrupole analyzers, ion trap analyzers, and time of flight analyzers. The ions may be detected using, for example, a selective mode or a scanning mode. Exemplary scanning modes include MRM and selected reaction monitoring (SRM).

Analysis results may include data produced by tandem MS. In exemplary embodiments, tandem MS may be accurate-mass tandem MS. For example, the accurate-mass tandem mass spectrometry may use a quadrupole time-of-flight (Q-TOF) analyzer. Tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion.

For example, a primary mass spectrum may contain five distinct ions, which may be represented as five graphical peaks. Each ion in the primary MS may be a parent ion. Each parent ion may be subjected to a secondary MS that produces a mass spectrum showing the daughter ions for that particular parent ion.

The parent/daughter relationship may be extended to describe the relationship between separated components (e.g., components eluting from the chromatography state) and ions detected in the primary MS, and to the relationship between the sample to be analyzed and the separated components.

The mass spectrometer typically provides the user with an ion scan (i.e., a relative abundance of each ion with a particular mass/charge over a given range). Mass spectrometry data may be related to the amount of the analyte in the original sample by a number of methods. In one example, a calibration standard is used to generate a standard curve (calibration curve) so that the relative abundance of a given ion may be converted into an absolute amount of the original analyte. In another example, the calibration standard may be an external standard and a standard curve may be generated based on ions generated from those standards to calculate the quantity of one more analytes. In a further example, the external standard may be an unlabeled analyte.

Internal standards may be added to calibration standards and/or test samples. An internal standard may be used to account for loss of analytes during sample processing in order to get a more accurate value of a measured analyte in the sample. The ratio of analyte peak area to internal standard peak area in the levels of the calibration standards may be used to generate a calibration curve and quantitate samples. One or more isotopically labeled analogs of analytes may be used as internal standards. In some embodiments, the analogs of analytes for use as internal standards may be labeled with deuterium, carbon 13 (¹³C), oxygen 17 (¹⁷O), oxygen 18 (¹⁸O), sulfur 33 (³³S), sulfur 34 (³⁴S), tritium (³H), carbon 14(¹⁴C), or a combination thereof. Non-limiting examples of labeled analogs that may be used as internal standards include trimethylamine N-oxide-¹³C₃, 3-indolepropionic acid-d₂, indole-d₇, N-acetylcadaverine-d₃, 5-aminovaleric acid-d₄, cadaverine-d₄, famotidine-¹³C₃, gamma-aminobutyric acid-d₆, serotonin-d₄, pipecolic acid-d₉, imidazole propionic acid-d₃, imidazolelactic acid-d₃, N-palmitoyl serinol-d₃, cylco(-His-Pro)-d₃, cyclo(-Pro-Thr)-d₃, cyclo(-Gly-His)-d₄, tryptophan-d₅, p-cresol-d₇, benzoic acid-d₅, hippurate-d₅, 4-hydroxyphenylacetic acid-d₆, 3-phenyllactic acid-d₅, (4-hydroxyphenyl)-2-propionic acid-d₆, naringenin-d₃, (3-phenylpropionyl)glycine-¹³C_{2,}¹⁵N₁, phenylacetylglycine-d₅, p-cresol sulfate-d₇, enterodiol-d₆, enterolactone-d₆, phenol sulfate-d₃, daidzein-d₄, apigenin-d₅, p-cresol glucuronide-d₇, genistein-d₄, ethylphenyl sulfate-d₄, equol-d₄, 3-indoxyl sulfate-¹³C₆, phenylacetic acid-d₇, deoxycholic acid-d₄, lithocholic acid-d₄, taurodeoxycholic acid-d₅, lactic acid-d₄, xylose-¹³C₅, raffinose-d₉, stachyose-d₇, diaminopimelic acid-¹³C_{7,}¹⁵N₂, trimethylamine-¹³C₃, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, urolithin B-¹³C₆, N-acetylmuramic acid-d₃, N-acetyl-D-neuraminic acid-1,2,3-¹³C₃, catechol sulfate-13C₆, or indolelactate-d₅. One or more isotopic labels may be added to the analogs of analytes used as internal standards. In some embodiments 2 or more, 3 or more, 4 or more, 5 or more, 6 or more, 7 or more, 8 or more, 9 or more, 10 or more isotopic labels may be added to the analog. In some embodiments, such as when a labeled analog of the analyte is not commercially available or cannot be synthesized, a structurally similar labeled compound may be used for quantitation. For example, the internal standard N-acetyl-D-neuraminic acid-1,2,3-¹³C₃ may be used for the quantitation of the analyte N-acetylmuraminate.

The analysis data from the MS instrument may be sent to a computer and processed using computer software. In one example, peak area ratios of analyte to internal standard are fitted against the concentrations of the calibration standards using a statistical regression method for quantitation. In another example, the statistical regression is weighted linear least squares regression. The slope and intercept calculated using the calibration curve may be used to calculate the unknown concentrations of analytes in experimental samples.

### IV. Kit

A kit for assaying one or more of the microbiome panel analytes or a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof, is described herein. For example, a kit may include packaging material and measured amounts of one or more calibration standards, analyte standards, internal standards, or quality control samples in amounts sufficient for one or more assays. In exemplary embodiments, the internal standards may be labeled (such as isotopically labeled or radiolabeled), the kit may comprise pre-made calibration standard solutions, internal standard solutions, mobile phase solutions, quality control samples, quality control sample reconstitution solutions, and/or the kit may comprise calibration standard reagents, internal standard reagents, mobile phase reagents, and instructions to prepare the mobile phase solutions. Kits may also comprise instructions recorded in tangible form (e.g. on paper such as, for example, an instruction booklet or an electronic medium) for using the reagents to measure the one or more analytes.

In exemplary embodiments, the one or more internal standards or a plurality of internal standards for use with the kit may include one or a plurality of internal standards selected from the group consisting of N-palmitoyl serinol-d₃, trimethylamine N-oxide-¹³C₃, 3-indolepropionic acid-d₂, indole-d₇, N-acetylcadaverine-d₃, 5-aminovaleric acid-d₄, cadaverine-d₄, famotidine-¹³C₃, gamma-aminobutyric acid-d₆, serotonin-d₄, pipecolic acid-d₉, imidazole propionic acid-d₃, imidazolelactic acid-d₃, cylco(-His-Pro)-d₃, cyclo(-Pro-Thr)-d₃, cyclo(-Gly-His)-d₄, tryptophan-d₅, p-cresol-d₇, benzoic acid-d₅, hippurate-d₅, 4-hydroxyphenylacetic acid-d₆, 3-phenyllactic acid-d₅, (4-hydroxyphenyl)-2-propionic acid-d₆, naringenin-d₃, (3-phenylpropionyl)glycine-¹³C_{2,}¹⁵N₁, phenylacetylglycine-d₅, p-cresol sulfate-d₇, enterodiol-d₆, enterolactone-d₆, phenol sulfate-d₃, daidzein-d₄, apigenin-d₅, p-cresol glucuronide-d₇, genistein-d₄, ethylphenyl sulfate-d₄, equol-d₄, 3-indoxyl sulfate-¹³C₆, phenylacetic acid-d₇, deoxycholic acid-d₄, lithocholic acid-d₄, taurodeoxycholic acid-d₅, lactic acid-d₄, xylose-¹³C₅, raffinose-d₉, stachyose-d₇, diaminopimelic acid-¹³C_{7,}¹⁵N₂, trimethylamine-¹³C₃, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, urolithin B-¹³C₆, N-acetylmuramic acid-d₃, N-acetyl-D-neuraminic acid-1,2,3-¹³C₃, catechol sulfate-¹³C₆, and indolelactate-d₅, and combinations thereof.

In other exemplary embodiments, the one or more internal standards for use with the kit may include one or a plurality of internal standards selected from the group consisting of N-acetyl-cadaverine-d₃, 5-aminovalerate-d₄, imidazole propionate-d₃, β-imidazolelactic acid-d₃, N-palmitoyl serinol-d₃, cylco(-His-Pro)-d₃, cyclo(-Pro-Thr)-d₃, cyclo(-Gly-His)-d₄, 2-(4-hydroxyphenyl)propionate-d₆, naringenin-d₃ sodium salt, phenol sulfate-d₃, ethylphenyl sulfate-d₄, raffinose-d₉, stachyose-d₇, 4-hydroxyphenyllactate-d₂, phenol glucuronide-d₅, N-acetylmuramic acid-d₃, catechol sulfate-¹³C₆, and combinations thereof.

In one embodiment, a kit for assaying one or more or a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, trimethylamine (TMA), and combinations thereof, is described herein. The internal standards for use with the kit may be selected from the group consisting of N-palmitoyl serinol-d₃, 3-indolepropionic acid-d₂, indole-d₇, tryptophan-d₅, 5-aminovaleric acid-d₄, pipecolic acid-d₉, N-acetylcadaverine-d₃, cadaverine-d₄, trimethylamine N-oxide-¹³C₃, gamma-aminobutyric acid-d₆, serotonin-d₄, imidazole propionic acid-d₃, imidazolelactic acid-d₃, cylco(-His-Pro)-d₃, cyclo(-Pro-Thr)-d₃, cyclo(-Gly-His)-d₄, famotidine-¹³C₃, diaminopimelic acid-¹³C_{7,}¹⁵N₂, and trimethylamine-¹³C₃.

In another embodiment, a kit for assaying one or more or a plurality of analytes selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, urolithin A, and combinations thereof, is described herein. The internal standards for use with the kit may be selected from the group consisting of p-cresol-d₇, 3-indoxyl sulfate-¹³C₆, 4-hydroxyphenylacetic acid-d₆, (4-hydroxyphenyl)-2-propionic acid-d₆, benzoic acid-d₅, phenylacetic acid-d₇, 3-phenyllactic acid-d₅, hippurate-d₅, lactic acid-d₄, (3-phenylpropionyl)glycine-¹³C_{2,}¹⁵N₁, phenylacetylglycine-d₅, ethylphenyl sulfate-d₄, phenol sulfate-d₃, p-cresol sulfate-d₇, p-cresol glucuronide-d₇, enterodiol-d₆, enterolactone-d₆, equol-d₄, daidzein-d₄, apigenin-d₅, naringenin-d₃, genistein-d₄, deoxycholic acid-d₄, lithocholic acid-d₄, and taurodeoxycholic acid-d₅, hydrocinnamic-d₅ acid, 4-ethylphenol-2,3,5,6-d₄,OD, 4-hydroxyphenyllactate-d₂, cinnamic-d₅ acid, cinnamoylglycine-2,2-d₂, phenol glucuronide-d₅, and urolithin B-¹³C₆.

In yet another embodiment, a kit for assaying one or more or a plurality of analytes selected from the group consisting of xylose, raffinose, stachyose, N-acetylmuraminate, N-acetylneuraminate (sialic acid), and combinations thereof, is described herein. The internal standards for use with the kit may be selected from the group consisting of xylose-¹³C₅, raffinose-d₉, stachyose-d₇, N-acetylmuramic acid-d₃, and N-acetyl-D-neuraminic acid-1,2,3-¹³C₃.

In a further embodiment, a kit for assaying one or more or a plurality of analytes selected from the group consisting of catechol sulfate, p-cresol sulfate, ethylphenyl sulfate, indole lactate, indolepropionate, indoxyl sulfate, and combinations thereof, is described herein. The internal standards for use with the kit may be selected from the group consisting of catechol sulfate-¹³C₆, p-cresol sulfate-d₇, ethylphenyl sulfate-d₄, indolelactate-d₅, indolepropionate-d₂, and 3-Indoxyl sulfate-¹³C₆.

### Examples

### I. Reagents and Instruments

HPLC grade methanol, ethanol, water and acetonitrile was obtained from Fisher Scientific. A Multi-Tube Vortexer from VWR Scientific was used for mixing. Centrifugation of plates was carried out in a Sorvall ST 40R centrifuge from Thermo Scientific with a 3617 bucket rotor. Reagents were obtained from commercial sources. Internal standards were obtained from commercial sources or were synthesized in-house.

### II. Internal Standard Synthesis

The following internal standards used in the methods described herein were not available from commercial sources and were synthesized in-house: N-acetyl-cadaverine-d₃, 5-aminovalerate-d₄, imidazole propionate-d₃, β-imidazolelactic acid-d₃, N-palmitoyl serinol-d₃, cylco(-His-Pro)-d₃, cyclo(-Pro-Thr)-d₃, cyclo(-Gly-His)-d₄, 2-(4-hydroxyphenyl)propionate-d₆, naringenin-d₃ sodium salt, phenol sulfate-d₃, ethylphenyl sulfate-d₄, raffinose-d₉, stachyose-d₇, 4-hydroxyphenyllactate-d₂, phenol glucuronide-d₅, N-acetylmuramic acid-d₃, and catechol sulfate-¹³C₆.

One of ordinary skill in the art will understand that the nomenclature used for deuterated compounds usually reflects the isomer with the highest incorporation of deuteration. However, in Hydrogen-Deuterium (H-D) exchange chemistry, incorporation of deuterium is usually not complete and results in a mixture of isomers. The distribution of isomers is noted for each synthesized compound. The deuteration level denoted in the names of the compounds below reflects the isomer that was used as an internal standard for the methods described herein. This isomer does not always reflect the isomer with the highest deuteration incorporation in the mixture.

### N-Acetyl-Cadaverine-d₃

To a stirring suspension of N-acetylcadaverine (27 mg, 0.187 mmol, 1.0 eq) in D₂O (0.50 mL) was added NaOD in D₂O (40 wt %, 0.100 mL). This mixture was stirred overnight at 45° C. but showed incomplete H-D exchange. An additional aliquot of NaOD in D₂O was added (˜0.100 mL) and stirring was continued overnight. When H-D exchange was judged complete by HRMS (d₀=<0.5%) the reaction mixture was neutralized to pH=7 with HCl and lyophilized to provide 25 mg (93%, crude) of N-acetyl-cadaverine-d₃, which was used without any further purification. The high resolution electrospray ionization mass spectrometry HRMS(ESI) mass to charge ratio (m/z) was calculated as 146.1378 for C₇H₁₃D₃N₂O+H]⁺. The m/z observed for synthesized N-acetyl-cadaverine-d₃ was 146.1385. The isotopic distribution as analyzed by HRMS(ESI) was d₀=0%, d₁=1%, d₂=11%, d₃=71%, d₄=6%, d₅=0%.

### 5-Aminovalerate-d₄

To a stirring suspension of bromopentanoic acid-d₄ (75.0 mg, 0.405 mmol, 1.0 eq) in ACN (0.200 mL) was added dibenzylamine (75.0, 0.380 mmol, 0.94 eq). The reaction mixture was stirred at 40° C. for several hours, and the solvent was removed. The residue was taken up in 1.5 mL EtOH and stirred with 10% Pd/C (20.0 mg) under an atmosphere of H₂ overnight. After filtering, the solvent was removed, and the residue was dissolved in water and lyophilized to provide 38 mg (78%, crude) of 5-aminovalerate-d₄ which was used without any further purification. The high-resolution electrospray ionization mass spectrometry HRMS(ESI) mass to charge ratio (m/z) was calculated as 120.0968 for C₅H₇D₄NO₂—H]⁻. The m/z observed for synthesized 5-aminovalerate-d₄ was 120.0973.

### Imidazole Propionate-d₃

To a stirring suspension of 4-imidazoleacrylic acid (20.0 mg) in D₂O (1.0 mL) was added 10% Pd/C (2.5 mg) and 10% Pt/C (2.5 mg). The reaction mixture was stirred under an atmosphere of H₂ overnight at 60° C. When H-D exchange was judged complete by HRMS (d₀=<0.5%) the reaction mixture was filtered and concentrated to provide 5 mg (25%, crude) of imidazole propionate-d₃, which was used without any further purification. The high resolution electrospray ionization mass spectrometry HRMS(ESI) mass to charge ratio (m/z) was calculated as 146.0764 for C₆H₄D₄N₂O₂—H]⁻. The m/z observed for synthesized imidazole propionate-d₃ was 143.0773. The isotopic distribution as analyzed by HRMS(ESI) was d₀=0%, d₁=1%, d₂=12%, d₃=26%, d₄=45%, d₅=17%.

### β-Imidazolelactic Acid-d₃

To a stirring suspension of histidine monohydrochloride monohydrate (75.0 mg, 0.358 mmol) in D₂O (2.5 mL) was added 10% Pd/C (2.5 mg) and 5% Pt/C (2.5 mg). The reaction mixture was stirred under an atmosphere of H₂ overnight at 60° C. When H-D exchange was judged complete by HRMS (d₀=<0.5%) the reaction mixture was filtered and concentrated to an oil which was used in the next step without any further purification. This residue was dissolved in 8:2 water/HOAc (1 mL) and treated with 2M NaNO₂ (49.0 mg (2.0 eq) dissolved in 0.35 mL water). After stirring overnight, the solvent was evaporated and the residue triturated with MeOH. The supernatant was filtered and dried to provide 45.0 mg (60%, crude) of β-imidazolelactic acid-d₃, which was used without any further purification. The high-resolution electrospray ionization mass spectrometry HRMS(ESI) mass to charge ratio (m/z) was calculated as 158.0650 for C₆H₅D₃N₂O₃—H]⁻. The m/z observed for synthesized β-imidazolelactic acid-d₃ was 158.0648. The isotopic distribution as analyzed by HRMS(ESI) was d₀=0%, d₁=5%, d₂=51%, d₃=36%, d₄=8%, d₅=1%.

### N-Palmitoyl Serinol-d₃

To a stirring suspension of hexadecenoic acid-₃ (25.0 mg, 0.0966 mmol, 1.0 eq), serinol (8.80 mg, 0.966 mmol, 1.0 eq), and HBTU (31.0 mg, 0.116 mmol, 1.2 eq) in DMF (1.0 mL) was added DIPEA (67.0 μL, 0.386 mmol, 1.2 eq). A precipitate immediately formed. After stirring overnight at RT, the reaction mixture was diluted with water, and the solids were collected by suction filtration to provide 17.9 mg of N-palmitoyl serinol-d₃ (56%), which was used without any further purification. The high-resolution electrospray ionization mass spectrometry HRMS(ESI) mass to charge ratio (m/z) was calculated as 331.3045 for C₁₉H₃₆D₃NO₃—H]⁻. The m/z observed for synthesized N-palmitoyl serinol-d₃ was 331.3045.

### Cylco(-His-Pro)-d₃

To a stirring suspension of Cyclo(-His-Pro) (25.0 mg) in D₂O (1.0 mL) was added 10% Pd/C (4.0 mg) and 5% Pt/C (4.0 mg). The reaction mixture was stirred under an atmosphere of H₂ for 48 h at 100° C. When H-D exchange was judged complete by HRMS (d₀=<0.5%) the reaction mixture was filtered and lyophilized to provide 19.0 mg (76%, crude) of cylco(-His-Pro)-d₃, which was used without any further purification. The high-resolution electrospray ionization mass spectrometry HRMS(ESI) mass to charge ratio (m/z) was calculated as 236.1232 for C₁₁H₁₁D₃N₄O₂—H]⁻. The m/z observed for synthesized cylco(-His-Pro)-d₃ was 236.1237. The isotopic distribution as analyzed by HRMS(ESI) was d₀=0%, d₁=3%, d₂=23%, d₃=47%, d₄=24%, d₅=3%.

### Cyclo(-Pro-Thr)-d₃

To a stirring suspension of Cyclo(-Pro-Thr) (25.0 mg) in D₂O (1.0 mL) was added 10% Pd/C (4.0 mg) and 5% Pt/C (4.0 mg). The reaction mixture was stirred under an atmosphere of H₂ for 48 h at 100° C. When H-D exchange was judged complete by HRMS (d₀=<0.5%) the reaction mixture was filtered and lyophilized to provide 19 mg (76%, crude) of cyclo(-Pro-Thr)-d₃, which now existed as a mixture of diastereomers. The compound was used without any further purification. The high-resolution electrospray ionization mass spectrometry HRMS(ESI) mass to charge ratio (m/z) was calculated as 202.1265 for C₉H₁₁D₃N₂O₃—H]⁻. The m/z observed for synthesized cyclo(-Pro-Thr)-d₃ was 202.1260. The isotopic distribution as analyzed by HRMS(ESI) was d₀=0%, d₁=1%, d₂=24%, d₃=39%, d₄=17%, d₅=10%, d₆=5%, d₇=3%.

### Cyclo(-Gly-His)-d₄

To a stirring suspension of Cyclo(-Gly-His) (25.0 mg) in D₂O (1.0 mL) was added 10% Pd/C (4.0 mg) and 5% Pt/C (4.0 mg). The reaction mixture was stirred under an atmosphere of H₂ for 48 h at 100° C. When H-D exchange was judged complete by HRMS (d₀=<0.5%) the reaction mixture was filtered and lyophilized to provide 18 mg (72%, crude) of cyclo(-Gly-His)-d₄, which was used without any further purification. The high resolution electrospray ionization mass spectrometry HRMS(ESI) mass to charge ratio (m/z) was calculated as 199.1128 for C₈H₆D₄N₄O₂+H]⁺. The m/z observed for synthesized cyclo(-Gly-His)-d₄ was 199.1117. The isotopic distribution as analyzed by HRMS(ESI) was d₀=0%, d₁=1%, d₂=9%, d₃=34%, d₄=40%, d₅=14%, d₆=1%.

### 2-(4-Hydroxyphenyl)Propionate-d₆

To a stirring suspension of (4-Hydroxyphenyl)-2-propionic acid (30.0 mg) in D₂O (2.0 mL) was added 10% Pd/C (2.0 mg) and 5% Pt/C (2.0 mg). The reaction mixture was stirred under an atmosphere of H₂ overnight in a sealed tube at 180° C. When H-D exchange was judged complete by HRMS (d₀=<0.5%) the reaction mixture was filtered and lyophilized to provide 23 mg (77%, crude) of 2-(4-hydroxyphenyl)propionate-d₆, which was used without any further purification. The high-resolution electrospray ionization mass spectrometry HRMS(ESI) mass to charge ratio (m/z) was calculated as 171.0934 for C₉H₄D₆O₃—H]⁻. The m/z observed for synthesized 2-(4-hydroxyphenyl)propionate-d₆ was 171.0939. The isotopic distribution as analyzed by HRMS(ESI) was d₀=0%, d₁=0%, d₂=10%, d₃=24%, d₄=13%, d₅=12%, d₆=32%, d₇=8%.

### Naringenin-d₃ Sodium Salt

To a stirring suspension of naringenin (30.0 mg) in D₂O (2.0 mL) was added 10% Pd/C (3.5 mg). The reaction mixture was stirred under an atmosphere of H₂ overnight in a sealed tube at 160° C. When H-D exchange was judged complete by HRMS (d₀=<0.5%) the reaction mixture was cooled and the product was precipitated. One drop of 6M NaOH was used to convert the product to the soluble Na+ salt and the mixture was filtered and lyophilized to provide 36.0 mg (113%, crude) of naringenin-d₃ sodium salt which was used without any further purification. The high-resolution electrospray ionization mass spectrometry HRMS(ESI) mass to charge ratio (m/z) was calculated as 274.0800 for C₁₅H₉D₃O₅—H]⁻. The m/z observed for synthesized naringenin-d₃ sodium salt was 274.0783. The isotopic distribution as analyzed by HRMS(ESI) was d₀=0%, d₁=2%, d₂=16%, d₃=67%, d₄=15%.

### Phenol Sulfate-d₃

To a stirring suspension of phen-2,4,6-d₃-ol (20.0 mg, 0.208 mmol, 1.0 eq) in pyridine (1.0 mL) was added SO₃-Pyr (38.0 mg, 0.239 mmol, 1.15 eq). The reaction mixture was stirred overnight at 60° C., cooled, and the solvent was removed under a stream of nitrogen to provide 41.0 mg (111%, crude) of phenol sulfate-d₃, which was used without any further purification. The high-resolution electrospray ionization mass spectrometry HRMS(ESI) mass to charge ratio (m/z) was calculated as 176.0102 for C₆H₃D₃O₄S—H]⁻. The m/z observed for synthesized phenol sulfate-d₃ was 176.0106.

### Ethylphenyl Sulfate-d₄

To a stirring suspension of 4-ethylphenol-2,3,5,6-d₄ (50.0 mg, 0.394 mmol, 1.0 eq) in pyridine (1.0 mL) was added SO₃—Pyr (93.0 mg, 0.591 mmol, 1.5 eq). The reaction mixture was stirred overnight at 60° C. and then for a week at room temperature. The solvent was removed under a stream of nitrogen. The solid was triturated with EtOAc, filtered, and the solvent was removed to provide 109 mg (134%, crude) of ethylphenyl sulfate-d₄, which was used without any further purification. The high-resolution electrospray ionization mass spectrometry HRMS(ESI) mass to charge ratio (m/z) was calculated as 205.0478 for C₈H₆D₄O₄S—H]⁻. The m/z observed for synthesized ethylphenyl sulfate-d₄ was 205.0479.

### Raffinose-d₉

To a stirring suspension of raffinose (20.0 mg) in D₂O (1.0 mL) was added 5% Ru/C (3.0 mg). The reaction mixture was stirred under an atmosphere of H₂ overnight at 80° C. When H-D exchange was judged complete by HRMS (d₀=<0.5%) the reaction mixture was filtered and lyophilized to provide 12.0 mg (80%, crude) of raffinose-d₉, which was used without any further purification. The high-resolution electrospray ionization mass spectrometry HRMS(ESI) mass to charge ratio (m/z) was calculated as 513.2182 for C₁₈H₂₃D₉O₁₆—H]⁻. The m/z observed for synthesized raffinose-d₉ was 513.2258. The isotopic distribution as analyzed by HRMS(ESI) was d₀=0%, d₁=0%, d₂=0%, d₃=0%, d₄=0%, d₅=0%, d₆=1%, d₇=5%, d₈=12%, d₉=21%, d₁₀=24%, d₁₁=19%, d₁₂=10%, d₁₃=4%, d₁₄=4%, d₁₅=1%.

### Stachyose-d₇

To a stirring suspension of stachyose (15.0 mg) in D₂O (1.0 mL) was added 5% Ru/C (3.0 mg). The reaction mixture was stirred under an atmosphere of H₂ overnight at 80° C. When H-D exchange was judged complete by HRMS (d₀=<0.5%) the reaction mixture was filtered and lyophilized to provide 12 mg (80%, crude) of stachyose-d₇, which was used without any further purification. The high-resolution electrospray ionization mass spectrometry HRMS(ESI) mass to charge ratio (m/z) was calculated as 672.2585 for C₁₈H₂₃D₉O₁₆—H]⁻. The m/z observed for synthesized stachyose-d₇ was 672.2600. The isotopic distribution as analyzed by HRMS(ESI) was d₀=0%, d₁=0%, d₂=0%, d₃=1%, d₄=4%, d₅=11%, d₆=17%, d₇=21%, d₈=18%, d₉=13%, d₁₀=7%, din=4%, d₁₂=2%.

Catechol Sulfate-¹³C₆

To a stirring solution of ¹³C₆ labelled catechol (2.5 mg, 0.02154 mmol, 1.0 eq) in pyridine (50 uL) SO₃—Pyr (3.8 mg, 0.02369 mmol, 1.1 eq) was added. The reaction mixture was stirred at 40° C. for several hours, but the starting material was not fully consumed. An additional aliquot of SO₃—Pyr (2.0 mg) was added with additional pyridine (˜200 uL), and the mixture was stirred overnight. Although some starting material remained, the reaction mixture was quenched with 1 M KOH (150 uL). This solution was added dropwise to 2-propanol (2 mL), the mixture was centrifuged, and the supernatant was decanted. This process was repeated twice, and the resultant residue was dried under vacuum to provide catechol sulfate-¹³C₆ (˜10 mg) which was used without any further purification. The HRMS(ESI) m/z calculated for ¹³C₆H₆O₂S—H]⁻ was 195.0064. The m/z observed for synthesized catechol sulfate-¹³C₆ was 195.0080.

### III. Internal Standard Preparation

Working internal standard (WIS) solutions were prepared at the concentrations indicated in Table 1 in water:acetonitrile (1:1). In some examples, a different WIS concentration may be needed for different sample types. One having skill in the art will understand how to determine the WIS for the given sample type.

TABLE 1
Working Internal Standard (WIS) Solutions

| | Concentration |
|---|---|
| Internal Standard Name | (μg/mL) |
| Trimethylamine N-oxide-¹³C₃ | 1 or 0.4 |
| 3-Indolepropionic acid-d₂ | 2.5 or 1.0 |
| Indole-d₇ | 500 |
| N-Acetylcadaverine-d₃ | 500 |
| 5-Aminovaleric acid-d₄ | 100 |
| Cadaverine-d₄ | 2.5 |
| Famotidine-¹³C₃ | 0.1 |
| gamma-Aminobutyric acid-d₆ | 10 |
| Serotonin-d₄ | 2 |
| Pipecolic acid-d₉ | 0.4 |
| Imidazole propionic acid-d₃ | 0.05 |
| Imidazolelactic acid-d₃ | 20 |
| N-Palmitoyl serinol-d₃ | 1 |
| Cylco(-His-Pro)-d₃ | 10 |
| Cyclo(-Pro-Thr)-d₃ | 25 |
| Cyclo(-Gly-His)-d₄ | 5 |
| Tryptophan-d₅ | 5 |
| p-Cresol-d₇ | 500 |
| Benzoic acid-d₅ | 1.25 |
| Hippurate-d₅ | 2 |
| 4-hydroxyphenylacetic acid-d₆ | 45 |
| 3-Phenyllactic acid-d₅ | 1 |
| (4-hydroxyphenyl)-2-propionic acid-d₆ | 160 |
| Naringenin-d₃ | 2 |
| (3-Phenylpropionyl)glycine-¹³C₂,¹⁵N₁ | 0.04 |
| Phenylacetylglycine-d₅ | 0.1 |
| p-Cresol sulfate-d₇ | 0.2 or 0.25 |
| Enterodiol-d₆ | 2.5 |
| Enterolactone-d₆ | 2.5 |
| Phenol sulfate-d₃ | 0.5 |
| Daidzein-d₄ | 1 |
| Apigenin-d₅ | 1 |
| p-Cresol glucuronide-d₇ | 0.25 |
| Genistein-d₄ | 1 |
| Ethylphenyl sulfate-d₄ | 0.1 or 0.02 |
| Equol-d₄ | 0.5 |
| 3-Indoxyl sulfate-¹³C₆ | 0.25 or 0.5 |
| Phenylacetic acid-d₇ | 120 |
| Deoxycholic acid-d₄ | 80 |
| Lithocholic acid-d₄ | 40 |
| Taurodeoxycholic acid-d₅ | 2 |
| Lactic acid-d₄ | 2 |
| Xylose-¹³C₅ | 40 |
| Raffinose-d₉ | 50 |
| Stachyose-d₇ | 50 |
| Catechol sulfate-¹³C₆ | 0.150 |
| Indolelactate-d₅ | 0.5 |

### IV. Synthesis of Calibration Standard

The Calibration standard for catechol sulfate used in the methods described herein was not available from a commercial source and was synthesized in-house.

### Catechol Sulfate, Dipotassium Salt

To a stirring solution of catechol (424 mg, 3.85 mmol, 1.0 eq) in pyridine (1.75 mL), SO₃-Pyr (665 mg, 4.20 mmol, 1.1 eq) was added. The reaction mixture was stirred at 40° C. for several hours and quenched with 1 M KOH (17.5 mL). Approximately half of this solution was added dropwise to 2-propanol. Initially, the product precipitated as a solid but eventually oiled out. The mixture was centrifuged and the supernatant was decanted. This trituration process was repeated twice, and the resultant residue was dried under vacuum to provide Catechol sulfate, dipotassium salt (˜200 mg), as a solid, which was used without any further purification. Quantitative NMR analysis showed the material was ˜36% pure, likely as the dipotassium salt in a mixture with potassium sulfate. The HRMS(ESI) m/z calculated for C₆H₆O₂S—H]⁻ was 188.9863. The m/z observed for catechol sulfate, dipotassium salt was 188.9860. ¹H NMR Spectrum (500 MHz; D₂O); δ(in ppm) 7.34-7.28 (dd, 1H); 7.05-7.01 (dt, 1H); 6.76-6.74 (dd, 1H), 6.56-6.53 (dt, 1H).

### V. Determination of Calibration Range

The calibration range of each analyte was determined using solutions spiked with known concentrations of calibration standards. For each analyte, the LLOQ represents the low end of the calibration range, and the high end of the calibration range is represented by the ULOQ.

In one example, to cover the calibration ranges in fecal samples, eight calibration standards (standards A-H in Table 2) were used.

TABLE 2
Calibration Ranges for Analytes

| | Concentration (μg/mL) | | | | | | | |
|---|---|---|---|---|---|---|---|---|
| Analyte | A | B | C | D | E | F | G | H |
| indolepropionate | 0.2 | 0.4 | 1 | 2 | 5 | 16 | 40 | 50 |
| indole | 4 | 8 | 20 | 40 | 100 | 320 | 800 | 1000 |
| tryptophan | 1.6 | 3.2 | 8 | 16 | 40 | 128 | 320 | 400 |
| 5-aminovalerate | 6 | 12 | 30 | 60 | 150 | 480 | 1200 | 1500 |
| pipecolate | 2 | 4 | 10 | 20 | 50 | 160 | 400 | 500 |
| N-acetyl-cadaverine | 8 | 16 | 40 | 80 | 200 | 640 | 1600 | 2000 |
| cadaverine | 16 | 32 | 80 | 160 | 400 | 1280 | 3200 | 4000 |
| trimethylamine-N- | 0.002 | 0.004 | 0.01 | 0.02 | 0.05 | 0.16 | 0.4 | 0.5 |
| oxide (TMAO) | | | | | | | | |
| gamma- | 0.8 | 1.6 | 4 | 8 | 20 | 64 | 160 | 200 |
| aminobutyric | | | | | | | | |
| acid (GABA) | | | | | | | | |
| serotonin | 0.2 | 0.4 | 1 | 2 | 5 | 16 | 40 | 50 |
| N-palmitoyl serinol | 0.008 | 0.016 | 0.04 | 0.08 | 0.2 | 0.64 | 1.6 | 2 |
| imidazole propionate | 0.0004 | 0.0008 | 0.002 | 0.004 | 0.01 | 0.032 | 0.08 | 0.1 |
| imidazole lactate | 0.04 | 0.08 | 0.2 | 0.4 | 1 | 3.2 | 8 | 10 |
| cyclo(-His-Pro) | 0.01 | 0.02 | 0.05 | 0.1 | 0.25 | 0.8 | 2 | 2.5 |
| cyclo(-Pro-Thr) | 0.1 | 0.2 | 0.5 | 1 | 2.5 | 8 | 20 | 25 |
| cyclo(-Gly-His) | 0.01 | 0.02 | 0.05 | 0.1 | 0.25 | 0.8 | 2 | 2.5 |
| famotidine | 0.004 | 0.008 | 0.02 | 0.04 | 0.1 | 0.32 | 0.8 | 1 |
| cresol | 40 | 80 | 200 | 400 | 800 | 1600 | 3200 | 4000 |
| 3-indoxyl sulfate | 0.004 | 0.008 | 0.02 | 0.04 | 0.1 | 0.32 | 0.8 | 1 |
| 4-hydroxyphenylacetate | 0.4 | 0.8 | 2 | 4 | 10 | 32 | 80 | 100 |
| 2-(4-hydroxyphenyl) | 16 | 32 | 80 | 160 | 400 | 1280 | 3200 | 4000 |
| propionate | | | | | | | | |
| benzoate | 1 | 2 | 5 | 10 | 20 | 40 | 80 | 100 |
| phenylacetic acid | 16 | 32 | 80 | 160 | 400 | 1280 | 3200 | 4000 |
| phenyllactate | 0.02 | 0.04 | 0.1 | 0.2 | 0.5 | 1.6 | 4 | 5 |
| hippurate | 0.04 | 0.08 | 0.2 | 0.4 | 1 | 3.2 | 8 | 10 |
| lactate | 4 | 8 | 20 | 40 | 100 | 320 | 800 | 1000 |
| phenylpropionylglycine | 0.001 | 0.002 | 0.005 | 0.01 | 0.025 | 0.08 | 0.2 | 0.25 |
| phenylacetylglycine | 0.0008 | 0.0016 | 0.004 | 0.008 | 0.02 | 0.064 | 0.16 | 0.2 |
| ethylphenyl sulfate | 0.00008 | 0.00016 | 0.0004 | 0.0008 | 0.002 | 0.0064 | 0.016 | 0.02 |
| phenol sulfate | 0.004 | 0.008 | 0.02 | 0.04 | 0.1 | 0.32 | 0.8 | 1 |
| p-cresol sulfate | 0.016 | 0.032 | 0.08 | 0.16 | 0.4 | 1.28 | 3.2 | 4 |
| p-cresol | 0.004 | 0.008 | 0.02 | 0.04 | 0.1 | 0.32 | 0.8 | 1 |
| glucuronide | | | | | | | | |
| enterodiol | 0.02 | 0.04 | 0.1 | 0.2 | 0.5 | 1.6 | 4 | 5 |
| enterolactone | 0.04 | 0.08 | 0.2 | 0.4 | 1 | 3.2 | 8 | 10 |
| equol | 0.004 | 0.008 | 0.02 | 0.04 | 0.1 | 0.32 | 0.8 | 1 |
| daidzein | 0.08 | 0.16 | 0.4 | 0.8 | 2 | 6.4 | 16 | 20 |
| apigenin | 0.08 | 0.16 | 0.4 | 0.8 | 2 | 6.4 | 16 | 20 |
| naringenin | 0.04 | 0.08 | 0.2 | 0.4 | 1 | 3.2 | 8 | 10 |
| genistein | 0.02 | 0.04 | 0.1 | 0.2 | 0.5 | 1.6 | 4 | 5 |
| deoxycholate | 50 | 100 | 250 | 500 | 1000 | 2000 | 4000 | 5000 |
| lithocholate | 50 | 100 | 250 | 500 | 1000 | 2000 | 4000 | 5000 |
| taurodeoxycholate | 0.1 | 0.2 | 0.5 | 1 | 2.5 | 8 | 20 | 25 |
| xylose | 2 | 4 | 10 | 20 | 50 | 160 | 400 | 500 |
| raffinose | 4 | 8 | 20 | 40 | 100 | 320 | 800 | 1000 |
| stachyose | 4 | 8 | 20 | 40 | 100 | 320 | 800 | 1000 |

In another example, to cover the calibration ranges in serum samples, the following calibration ranges were used: 1.00-400 ng/mL for 4-ethylphenyl sulfate, 5.00-2000 ng/mL for p-cresol sulfate, 10.0-4000 ng/mL for 3-indoxyl sulfate, 10.0-4000 ng/mL for catechol sulfate, 10.0-4000 ng/mL for indolelactate, 5.00-2000 ng/mL for indolepropionate, and 7.50-3000 ng/mL for trimethylamine oxide (TMAO).

One of ordinary skill in the art would understand how to determine the calibration range for additional analytes and/or sample types without undue experimentation. Calibration spiking solutions may be prepared at 100- or 250-fold of the corresponding calibration concentrations. The spiking solutions are used to produce Combined Calibration Standards for the analytical runs.

### VI. Sample Preparation

### Solid Samples

Approximately 20 mg of Experimental sample material (human fecal material) was weighed into a 1.5 mL tube, and the exact weight was recorded. Some solid samples, such as fecal samples, may require lyophilization to dry the sample prior to weighing it.

QC samples for feces were prepared by pooling fecal samples and fortifying with the analyte(s) or diluting with PBS or water, as needed, to obtain the desired analyte levels. Prior to use, all QC samples were stored at −80° C.

For Calibration Standards, Blank, and Blank-IS samples, 20.0 μL of water was added to 1.5 mL tubes. For QC samples, approximately 20.0 mg of QC sample material corresponding to the Experimental sample type was added to a 1.5 mL tube, and the exact weight was recorded. For Combined Calibration Standards, an 80.0 μL volume of Calibration Solution, corresponding to the Calibration Range determined for each analyte to be measured, was added to corresponding Combined Calibration Standards tubes (e.g., A-H), and an 80.0 μL volume of Ethanol/Acetonitrile/Water (2:1:1) was added to Standard, Blank-IS, QC and Experimental samples. A 20.0 μL volume of the WIS solution was added to the Combined Calibration Standard, Blank-IS, QC, and Experimental samples, and 20.0 μL of Acetontrile/Water (1:1) was added to the blank samples.

### Liquid Samples

50.0 μl of Experimental Sample (cat serum) was added to a well of a microtiter plate. QC samples for the serum were prepared by pooling twelve serum lots and taking aliquots of the pooled sample; analytes were at endogenous levels. All QC samples were stored at −80° C. until used for analysis.

For Blank and Blank-IS Samples, 50.0 μL of water was added to a well of a microtiter plate. For Calibration Standards, 50.0 μL of corresponding Calibration Solutions was added to a well of a microtiter plate. For QC Samples, 50.0 μL of QC sample material for the corresponding sample type was added to a well of a microtiter plate. A 20.0 μL volume of the WIS solution was added to Calibration Standard, Blank-IS, QC Samples, and Experimental Samples, and 20.0 μL of water was added to the blank samples.

### VII. Extraction

For Chromatography Methods 1-3, proteins were precipitated and analytes were extracted by adding 200 μL 1% Formic Acid in 70% Methanol to all samples, and the samples were shaken or vortexed for 5 minutes followed by centrifuging for 5 minutes at 4000 rpm. A 150 μL volume of cleared supernatant was transferred into a fresh 96-well plate. Plates were capped and subjected to LC-MS/MS analysis.

For Chromatography Methods 4 and 5, 40 μL ACN/Water was added to blank samples, and 20 μL ACN/Water was added to Blank-IS, QC, and Experimental samples. Proteins were precipitated and analytes were extracted by adding 200 μL of methanol to all samples, and samples were shaken or vortexed and then centrifuged. A 100 μL volume of cleared supernatant was transferred into a fresh 96-well plate. Plates were capped and subject to LC-MS/MS analysis.

### Example 1: Chromatographic Purification and Separation of Analytes from Samples

Chromatographic methods were developed using UHPLC to analyze up to fifty-eight analytes. Analytes were divided into panels, each panel having a separate chromatographic method.

For each chromatographic method a single fixed aliquot of 1.0 μL of the final extraction solution was injected onto the UPLC column for each sample analyzed. An Agilent 1290 Infinity UHPLC system equipped with a binary solvent pump unit, a refrigerated autosampler (set at 4° C.), and a column heater (set at 60° C. for Chromatography Methods 1-3 and 50° C. for Chromatography Methods 4 & 5) was used for liquid chromatography with a reversed phase column (Waters ACQUITY BEH C18, 1.7 μm, 2.1×100 mm) for Chromatography Methods 1, 2, & 4, a HILIC column (XBridge BEH Amide, 2.5 micron 2.1×100 mm) for Chromatography Method 3, and a Waters Acquity BEH Amide (1.7 micron, 2.1×150 mm) column for Chromatography Method 5. Each chromatography method is further exemplified below.

### A. Chromatography Method 1

In one example, a liquid chromatography method was developed for the purification and separation of a panel of up to nineteen analytes consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), in the same injection. The amounts of one or a plurality of analytes (e.g., two or more, three or more and up to nineteen analytes and combinations thereof selected from the panel) may be measured using this method.

Mobile phase A was PFPA in water and mobile phase B was PFPA in acetonitrile. Linear gradient elution was carried out with an initial condition of 0.5% mobile phase B (99.5% mobile phase A) and 600 μL/min flow rate. The total run time, including chromatography and mass spectrometry, was 7.00 min.

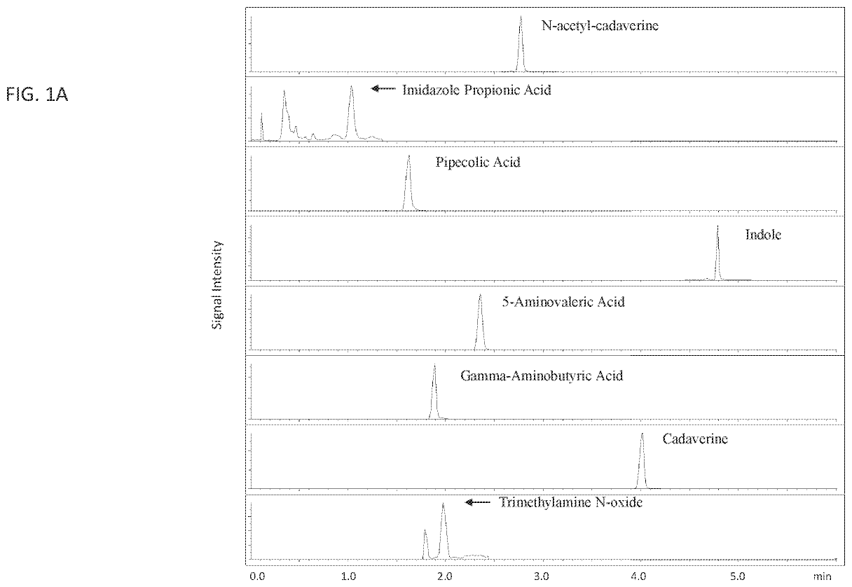

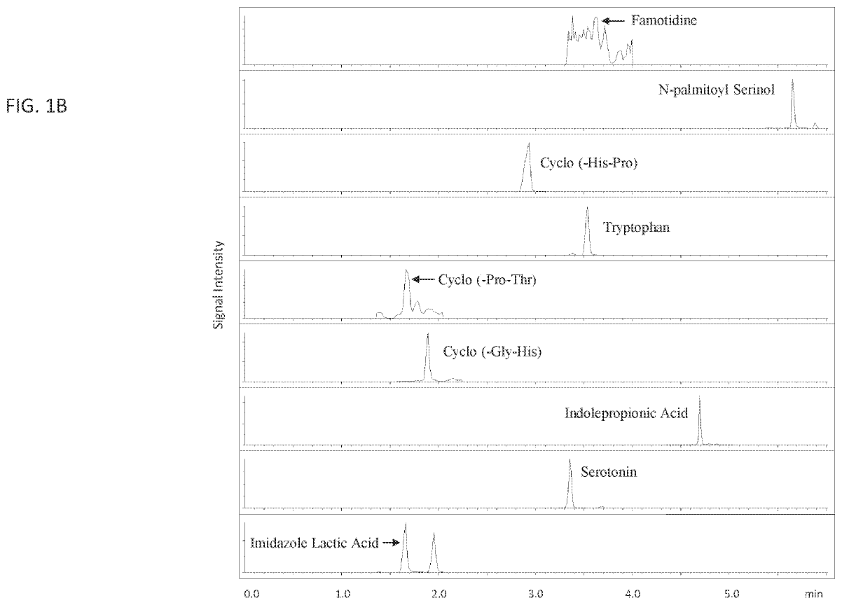

In one example of Chromatography Method 1, fecal samples were prepared as indicated above. A single fixed aliquot of 1.0 μL of the final analytical sample was injected onto the chromatography column for each sample analyzed. In this example, Chromatography Method 1 separated a plurality of up to seventeen analytes with good peak shapes. Exemplary chromatograms of the resulting separated analytes N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), and famotidine are shown in FIG. 1 (A-B) for fecal samples. Approximate retention times (in minutes) are shown in Table 3.

### B. Chromatography Method 2

In another example, a liquid chromatography method was developed for the purification and separation of a panel of up to thirty-two analytes consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, in the same injection. The amounts of one or a plurality of analytes (e.g., two or more, three or more and up to thirty-two analytes and combinations thereof selected from the panel) may be measured using this method.

Mobile phase A was formic acid in water and mobile phase B was formic acid in acetonitrile. Linear gradient elution was carried out with an initial condition of 0% mobile phase B (100% mobile phase A) and 600 μL/min flow rate. The total run time, including chromatography and mass spectrometry, was 9.00 min.

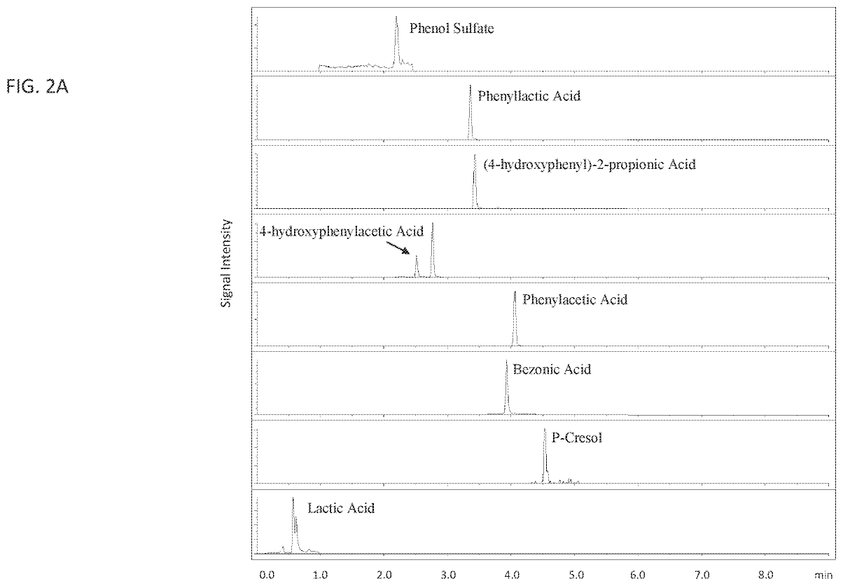

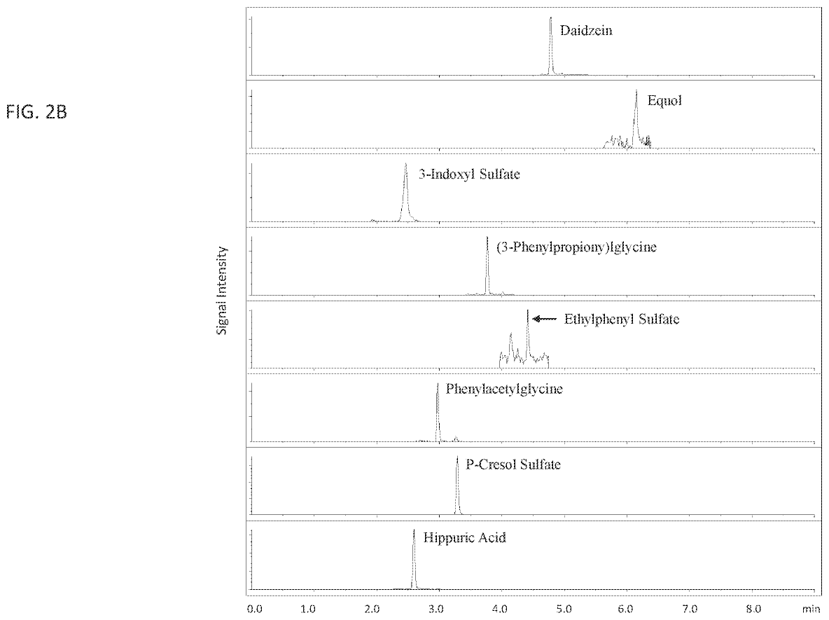

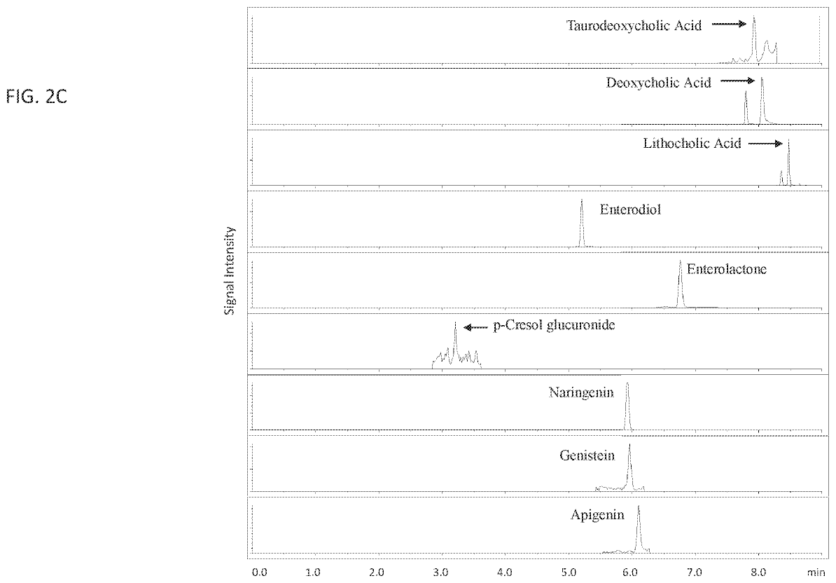

In one example of Chromatography Method 2, fecal samples were prepared as indicated above. A single fixed aliquot of 1.0 μL of the final analytical sample was injected onto the chromatography column for each sample analyzed. In this example, Chromatography Method 2 separated a plurality of up to twenty-five analytes with good peak shapes. Exemplary chromatograms of the resulting separated analytes cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, and taurodeoxycholate are shown in FIG. 2 (A-C) for fecal samples. Approximate retention times (in minutes) are shown in Table 4.

### C. Chromatography Method 3

In another example, a liquid chromatography method was developed for the purification and separation of a panel of up to five analytes consisting of xylose, raffinose, stachyose, N-acetylmuraminate, and N-acetylneuraminate (sialic acid), in the same injection. The amounts of one or a plurality of analytes (e.g., two or more, three or more, and up to five analytes and combinations thereof selected from the panel) may be measured using this method.

Mobile phase A was triethylamine in water and mobile phase B was triethylamine in acetonitrile. Linear gradient elution was carried out with an initial condition of 2% mobile phase A (98% mobile phase B) and 600 μL/min flow rate. The total run time, including chromatography and mass spectrometry, was 6.00 min.

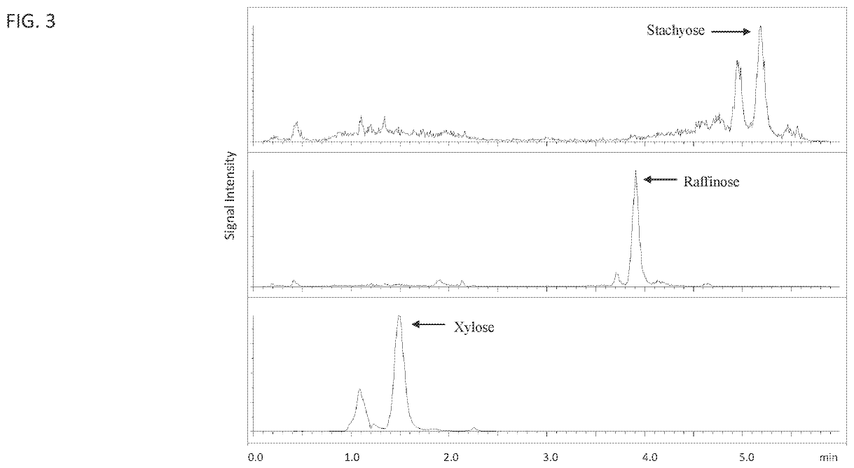

In one example of Chromatography Method 3, fecal samples were prepared as indicated above. A single fixed aliquot of 1.0 μL of the final analytical sample was injected onto the chromatography column for each sample analyzed. In this example, Chromatography Method 3 separated a plurality of up to three analytes with good peak shapes. Exemplary chromatograms of the resulting separated analytes xylose, raffinose, and stachyose are shown in FIG. 3 for fecal samples. Approximate retention times (in minutes) are shown in Table 5.

### D. Chromatography Method 4

In another example, a liquid chromatography method was developed for the purification and separation of a panel of up to six analytes consisting of catechol sulfate, p-cresol sulfate, ethylphenyl sulfate, indole lactate, indolepropionate, and indoxyl sulfate, in the same injection. The amounts of one or a plurality of analytes (e.g., two or more, three or more, and up to six analytes and combinations thereof selected from the panel) may be measured using this method.

Mobile phase A was formic acid in water and mobile phase B was formic acid in acetonitrile. Linear gradient elution was carried out with an initial condition of 10% mobile phase B (90% mobile phase A) and 550 μL/min flow rate. The total run time, including chromatography and mass spectrometry, was 5.50 min.

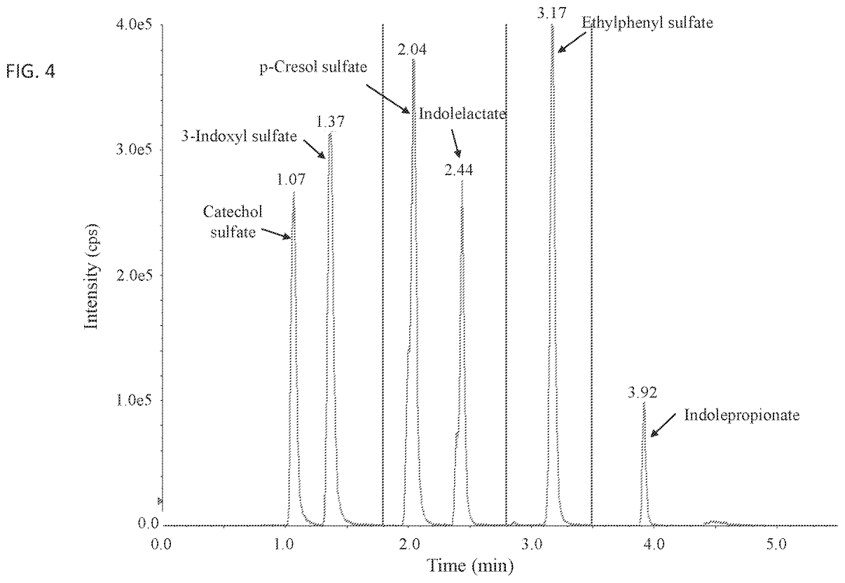

In one example of Chromatography Method 4, serum samples were prepared as indicated above. A single fixed aliquot of 1.0 μL of the final analytical sample was injected onto the chromatography column for each sample analyzed. Chromatography Method 4 separated a plurality of up to six analytes with good peak shapes. Exemplary chromatograms of the resulting separated analytes catechol sulfate, p-cresol sulfate, ethylphenyl sulfate, indole lactate, indolepropionate, and indoxyl sulfate are shown in FIG. 4. Approximate retention times (in minutes) are shown in Table 6.

### E. Chromatography Method 5

In another example, a liquid chromatography method was developed for the purification and separation of trimethylamine-N-oxide (TMAO). The amount of trimethylamine-N-oxide (TMAO) may be measured using this method.

Mobile phase A was ammonium formate in water and mobile phase B was ammonium formate in acetonitrile/water. Linear gradient elution was carried out with an initial condition of 5% mobile phase A (95% mobile phase B) and 550 μL/min flow rate. The total run time, including chromatography and mass spectrometry, was 4.30 min.

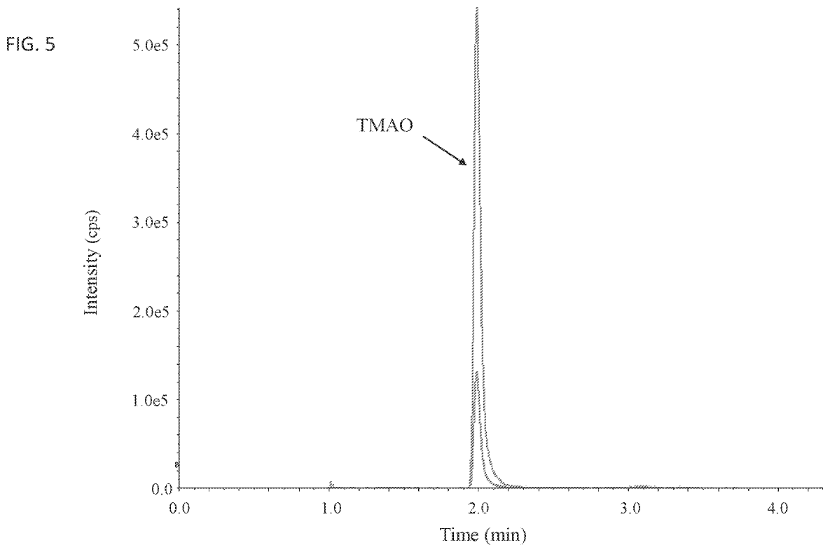

In one example of Chromatography Method 5, serum samples were prepared as indicated above. A single fixed aliquot of 1.0 μL of the final analytical sample was injected onto the chromatography column for each sample analyzed. Chromatography Method 5 separated trimethylamine-N-oxide (TMAO) with good peak shape. An exemplary chromatogram of trimethylamine-N-oxide (TMAO) is shown in FIG. 5, and the approximate retention time (in minutes) is shown in Table 4.

### Example 2: Mass Spectrometry Measurement of Analytes

Mass spectrometry was performed on the sample extracts as described in the methods below using an AB Sciex QTrap 6500 mass spectrometer with Turbo V source (ESI). Raw data were acquired from the instrument and processed using Analyst 1.6.2 software (AB Sciex). For quantitation, peak area ratios of analyte to internal standard were fitted against the concentrations of the calibration standards by weighted (1/×) linear or quadratic regression. The resulting slope and intercept of the calibration curve were used to calculate the unknown concentrations in experimental samples.

### A. MS Method 1

An MS method was developed to detect and determine the amounts of analytes. In the method (MS Method 1), the instrument was operated in positive multiple reaction monitoring (MRM) mode. Ionspray voltage was set at 5.5 kV, source temperature at 500° C., curtain gas (e.g., nitrogen) at 35 psi, and nebulizer and desolvation gas (e.g., nitrogen) flow rates at 70 psi, collisionally activated dissociation (CAD) gas (e.g., nitrogen) at medium. Methanol was used for needle wash.

MS Method 1 may be used to detect and determine the amounts of a panel of analytes consisting of N-palmitoyl serinol, indolepropionate, indole, tryptophan, 5-aminovalerate, pipecolate, N-acetyl-cadaverine, cadaverine, trimethylamine-n-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), in a single injection. The amounts of one or a plurality of analytes (e.g., two or more, three or more and up to nineteen analytes and combinations thereof selected from the panel) may be measured using this method.

In one example, MS Method 1 was used with Chromatography Method 1 to determine the amount of a panel of analytes. In this example, the eluent from the chromatography column described in Example 1, Chromatography Method 1, was directly and automatically introduced into the electrospray source of the mass spectrometer.

Exemplary ions that were generated for the quantitation of indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, N-palmitoyl serinol, imidazole propionate, imidazole lactate, cyclo(-His-Pro), cyclo(-Pro-Thr), cyclo(-Gly-His), and famotidine using Chromatography Method 1/MS Method 1 are listed in Table 3. The parent ions are listed under the column headed “Parent ion (m/z)”, and the daughter ions are listed in the column labeled “Daughter ion (m/z)”. The choice of daughter ion for quantitation in this example was optimized for sensitivity across the analytical measurement range; however, additional daughter ions may be selected to replace or augment the daughter ions used for quantitation in the examples.

TABLE 3
Parent and Daughter Ion Mass-to-Charge Ratios (m/z) of Analytes from
Chromatography Method 1 and MS Method 1

| | | | Approx- |
|---|---|---|---|
| | | | imate |
| | Parent Ion | Daughter Ions | Retention |
| Analyte | (m/z, ±0.5) | (m/z, ±0.5) | Time (min) |
| indolepropionate | 190.1 | 130.1; 128.1; 117.1; | 4.7 |
| | | 103.1; 77.1; 55.1 | |
| 3-indolepropionic | 192.1 | 174.1; 146.1; 130.1; | 4.7 |
| acid-d₂ | | 103.1; 77.1; 56.1 | |
| indole | 118.2 | 91.1; 65.1; 58.1 | 4.8 |
| indole-d₇ | 124.2 | 96.1; 69.1; 68.1 | 4.8 |
| tryptophan | 205.1 | 188.1; 170.1; 159.1; | 3.6 |
| | | 146.1; 144.1; 118.1 | |
| tryptophan-d₅ | 210.1 | 122.1; 147.1; 150.1; | 3.6 |
| | | 192.1 | |
| 5-aminovalerate | 118.1 | 101.1; 83.1; 59.1; 55.1 | 2.4 |
| 5-iminovaleric | 122.1 | 105.1; 91.1; 86.1; 79.1; | 2.4 |
| acid-d₄ | | 59.1 | |
| pipecolate | 130.2 | 84.1; 67.1; 56.1 | 1.6 |
| pipecolic acid-d₉ | 139.1 | 61.1; 75.1; 89.1; 93.1 | 1.6 |
| N-acetyl-cadaverine | 145.1 | 128.1; 86.1; 69.1; 60.1; | 2.8 |
| | | 41.1 | |
| N-acetyl- | 148.1 | 131.1; 87.1; 86.1; 75.1; | 2.8 |
| cadaverine-d₃ | | 69.1; 63.1 | |
| cadaverine | 103.2 | 86.1; 69.2; 41.1 | 4.0 |
| cadaverine-d₄ | 107.1 | 90.1; 73.1; 43.1 | 4.0 |
| trimethylamine- | 76.1 | 58.1; 42.1; 30.1 | 2.0 |
| N-oxide (TMAO) | | | |
| trimethylamine | 85.1 | 68.1; 66.1; 48.1; 46.1 | 2.0 |
| N-oxide-¹³C₃ | | | |
| gamma-aminobutyric | 104.1 | 87.1; 69.1; 45.1; 43.1 | 1.9 |
| acid (GABA) | | | |
| gamma-aminobutyric | 110.1 | 93.1; 73.1; 72.1; 49.1; | 1.9 |
| acid-d₆ | | 46.1; 45.1 | |
| serotonin | 177.1 | 160.2; 142.1; 132.1; | 3.3 |
| | | 117.1; 115.1 | |
| serotonin-d₄ | 181.1 | 164.1; 147.1; 136.1; | 3.3 |
| | | 118.1; 109.1 | |
| N-palmitoyl serinol | 330.3 | 312.1; 239.1; 149.1; | 5.7 |
| | | 139.1; 92.1; 74.1 | |
| N-palmitoyl | 333.3 | 315.3 | 5.7 |
| serinol-d₃ | | | |
| imidazole | 141.1 | 123.1; 95.1; 81.1; | 1.0 |
| propionate | | 69.1; 45.1; 42.1 | |
| imidazole propionic | 144.1 | 126.1; 98.1; 84.1; | 1.0 |
| acid-d₃ | | 83.1; 72.1; 45.1 | |
| imidazole lactate | 157.1 | 139.1; 111.1; 95.1; | 1.7 |
| | | 82.1; 56.1 | |
| β-imidazolelactic | 160.1 | 85.1; 114.1; 132.1 | 1.7 |
| acid-d₃ | | | |
| cyclo(-His-Pro) | 235.1 | 70.1; 110.1; 138.1; 162.1; | 3.0 |
| | | 166.1; 207.1; 217.1 | |
| cylco(-His-Pro)-d₃ | 238.1 | 210.1; 164.1; 113.1; | 3.0 |
| | | 112.1; 71.1 | |
| cyclo(-Pro-Thr) | 199.2 | 181.1; 171.1; 153.1; 125.1; | 1.7 |
| | | 97.1; 84.1; 74.1; 70.1 | |
| cyclo(-Pro-Thr)-d₃ | 202.1 | 156.1 | 1.7 |
| cyclo(-Gly-His) | 195.1 | 178.1; 167.1; 150.1; 122.1; | 1.9 |
| | | 110.1; 95.1; 82.1 | |
| cyclo(-Gly-His)-d₄ | 199 | 125 | 1.9 |
| famotidine | 338.1 | 259.1; 242.1; 189.1; 155.1; | 3.6 |
| | | 138.1 | |
| famotidine-¹³C₃ | 341.1 | 262.1; 192.1; 158.1; 141.1; | 3.6 |
| | | 115.1; 71.1 | |

In another example, MS Method 1 was used with Chromatography Method 5 to detect and determine the amount of trimethylamine-N-oxide (TMAO). In this example, the eluent from the chromatography column described in Example 1, Chromatography Method 5, was directly and automatically introduced into the electrospray source of the mass spectrometer.

Exemplary ions that were generated for the quantitation of trimethylamine-N-oxide (TMAO) using Chromatography Method 5/MS Method 1 are listed in Table 4.

TABLE 4
Parent and Daughter Ion Mass-to-Charge Ratios (m/z) of Analytes
from Chromatography Method 5 and MS Method 1

| | | Daughter | Approximate |
|---|---|---|---|
| | Parent Ion | Ion | Retention |
| Analyte | (m/z, ±0.5) | (m/z, ±0.5) | Time (min) |
| trimethylamine-N-oxide | 76 | 41.95 | 2.0 |
| (TMAO) | | | |
| trimethylamine N-oxide-¹³C₃ | 79 | 44 | 2.0 |

### B. MS Method 2

An MS method (MS Method 2) was developed to detect and determine the amounts of analytes. For this method, the MS instrument was operated in negative MRM mode. Ionspray voltage was set at −4.5 kV, source temperature at 500° C., curtain gas (e.g., nitrogen) at 35 psi, and nebulizer and desolvation gas (e.g., nitrogen) flow rates at 70 psi, collisionally activated dissociation (CAD) gas (e.g., nitrogen) at medium. Methanol was used for needle wash.

In one example, MS Method 2 was used with Chromatography Method 2 to detect and determine the amounts of a panel of analytes consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate 3-phenylpropionate, 4-ethylphenol, 4-hydroxyphenyllactate, cinnamate, cinnamoylglycine, phenol glucuronide, and urolithin A, in a single injection.

In this example, the eluent from the chromatography column described in Example 1, Chromatography Method 2, was directly and automatically introduced into the electrospray source of a mass spectrometer.

Exemplary ions that were generated for the quantitation of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, and taurodeoxycholate using Chromatography Method 2/MS Method 2 are listed in Table 5.

TABLE 5
Parent and Daughter Ion Mass-to-Charge Ratios (m/z) of Analytes from
Chromatography Method 2 and MS Method 2

| | | | Approxi- |
|---|---|---|---|
| | Parent | | mate |
| | Ion | Daughter Ions | Retention |
| Analyte | (m/z, ±0.5) | (m/z, ±0.5) | Time (min) |
| cresol | 107.1 | 92.1; 77.2; 45.1 | 4.55 |
| p-Cresol-d₇ | 114.1 | 96.1 | 4.55 |
| 3-indoxyl sulfate | 212.1 | 132.2; 104.1; 92.1; 80.1; | 2.38 |
| | | 77.1 | |
| 3-Indoxyl sulfate-¹³C₆ | 218.1 | 138.2 | 2.38 |
| 4-hydroxyphenylacetate | 151.1 | 136.1; 107.1; 93.1; 79.1 | 2.55 |
| 4-hydroxyphenylacetic | 157.1 | 113.1 | 2.55 |
| acid-d₆ | | | |
| 2-(4-hydroxy- | 165.1 | 93.1; 103.1; 106.1; | 3.44 |
| phenyl)propionate | | 119.1; 121.1; 149.1 | |
| (4-hydroxyphenyl)-2- | 171.1 | 153.1; 127.1; 124.1; | 3.44 |
| propionic acid-d₆ | | 103.1; 99.1; 95.1 | |
| benzoate | 121.1 | 59.1; 77.1; 93.1 | 4.00 |
| Benzoic acid-d₅ | 126.1 | 82.1 | 4.00 |
| phenylacetic acid | 135.2 | 91.1; 80.1 | 4.07 |
| Phenylacetic acid-d₇ | 142.2 | 98.1 | 4.07 |
| phenyllactate | 165.2 | 147.1; 119.1; 117.1; | 3.39 |
| | | 103.1; 91.1; 77.1; 73.1 | |
| 3-Phenyllactic acid-d₅ | 170.2 | 152.1 | 3.39 |
| hippurate | 178.2 | 134.1; 132.1; 116.1; | 2.63 |
| | | 77.1; 56.1; | |
| Hippurate-d₅ | 183.2 | 139.1 | 2.63 |
| lactate | 89.1 | 71.1; 45.1 | 0.56 |
| Lactic acid-d₄ | 93.1 | 46.1 | 0.56 |
| phenylpropionyl- | 206.2 | 162.1; 131.1; 103.1; | 3.80 |
| glycine | | 84.1; 74.1 | |
| (3-Phenylpropionyl) | 209.2 | 77.1 | 3.80 |
| glycine-¹³C₂,¹⁵N₁ | | | |
| phenylacetylglycine | 192.1 | 117.1; 91.1; 74.1; 46.1 | 2.99 |
| Phenylacetylglycine-d₅ | 197.1 | 74.1 | 2.99 |
| ethylphenyl sulfate | 201.1 | 121.1; 106.1; 80.1 | 4.40 |
| Ethylphenyl sulfate-d₄ | 205.1 | 125.1 | 4.40 |
| phenol sulfate | 173.1 | 93.1; 80.1 | 2.16 |
| Phenol sulfate-d₃ | 176.1 | 96 | 2.16 |
| p-cresol sulfate | 187.1 | 107.1; 80.1; 77.1 | 3.21 |
| p-Cresol sulfate-d₇ | 194.1 | 114.1 | 3.21 |
| p-cresol glucuronide | 283.3 | 175.1; 157.1; 113.1; | 3.23 |
| | | 107.1; 85.1 | |
| p-Cresol glucuronide-d₇ | 290.3 | 175.1 | 3.23 |
| enterodiol | 301.2 | 283.1; 271.1; 253.2; | 5.20 |
| | | 241.1; 146.1; 106.1; | |
| Enterodiol-d₆ | 307.2 | 258.2 | 5.20 |
| enterolactone | 297.1 | 253.1; 189.1; 165.1; | 6.80 |
| | | 145.1; 121.1; 107.1; | |
| Enterolactone-d₆ | 303.1 | 259.1 | 6.80 |
| equol | 241.2 | 147.1; 135.1; 121.1; | 6.10 |
| | | 119.1; 93.1; 91.1 | |
| Equol-d₄ | 245.2 | 121.1 | 6.10 |
| daidzein | 253.1 | 223.1; 208.1; 195.1; | 4.80 |
| | | 180.1; 132.1; 91.1 | |
| Daidzein-d₄ | 257.1 | 212.1 | 4.80 |
| apigenin | 269.1 | 107.1; 117.1; 149.1; | 6.05 |
| | | 151.1; 159.1; 227.1 | |
| Apigenin-d₅ | 274.1 | 120 | 6.05 |
| naringenin | 271.1 | 229.1; 187.1; 177.1; | 5.90 |
| | | 151.1; 119.1; 107.1; | |
| Naringenin-d₃ | 274.1 | 153.1 | 5.90 |
| | | 239.1; 223.1; 201.1; | |
| genistein | 269.3 | 180.1; 159.1; 133.2; | 5.95 |
| | | 107.1 | |
| Genistein-d₄ | 273.3 | 137.2 | 5.95 |
| | | 373.3; 355.3; 347.4; | |
| deoxycholate | 391.2 | 345.3; 343.4; 329.2; | 8.10 |
| | | 327.4; 311.3 | |
| Deoxycholic acid-d₄ | 395.2 | 349.3 | 8.10 |
| lithocholate | 375.3 | 375.1; 357.3; 301.1; | 8.40 |
| | | 183.1; 45.1 | |
| Lithocholic acid-d₄ | 379.3 | 379.3 | 8.40 |
| taurodeoxycholate | 497.7 | 80.1; 107.1; 124.1; | 7.90 |
| | | 355.4; 497.7 | |
| Taurodeoxycholic | 503.2 | 80.1 | 7.90 |
| acid-d₅ | | | |

In another example, MS Method 2 was used with Chromatography Method 4 to detect and determine the amounts of a panel of analytes consisting of catechol sulfate, p-cresol sulfate, ethylphenyl sulfate, indole lactate, indolepropionate, and indoxyl sulfate.

The eluent from the chromatography column described in Example 1, Chromatography Method 4, was directly and automatically introduced into the electrospray source of a mass spectrometer.

Exemplary ions that were generated for the quantitation of catechol sulfate, p-cresol sulfate, ethylphenyl sulfate, indole lactate, indolepropionate, and indoxyl sulfate using Chromatography Method 4/MS Method 2 are listed in Table 6.

TABLE 6
Parent and Daughter Ion Mass-to-Charge Ratios (m/z) of
Analytes from Chromatography Method 4 and MS Method 2

| | | | Approximate |
|---|---|---|---|
| | Parent Ion | Daughter Ions | Retention |
| Analyte | (m/z, ±0.5) | (m/z, ±0.5) | Time (min) |
| 3-indoxyl sulfate | 212 | 80.95 | 1.4 |
| 3-Indoxyl sulfate-¹³C₆ | 218.1 | 138.2 | 1.4 |
| catechol sulfate | 188.95 | 108.0 | 1.1 |
| catechol sulfate-¹³C₆ | 195 | 114.0 | 1.1 |
| p-cresol sulfate | 187 | 107.05 | 2.1 |
| p-cresol sulfate-d₇ | 194 | 114.05 | 2.1 |
| indolelactate | 204.05 | 128.0 | 2.4 |
| indolelactate-d₅ | 209.1 | 132.0 | 2.4 |
| ethylphenyl sulfate | 201 | 121.05 | 3.3 |
| ethylphenyl sulfate-d₄ | 205 | 125.05 | 3.3 |
| indolepropionate | 188.05 | 59 | 3.9 |
| indolepropionate-d₂ | 190 | 61 | 3.9 |

### C. MS Method 3

Another MS method (MS Method 3) was developed to detect and determine the amounts of a panel of analytes consisting of xylose, raffinose, stachyose, N-acetylmuraminate, and N-acetylneuraminate (sialic acid), in a single injection. The amounts of one or a plurality of analytes (e.g., two or more, three or more and up to five analytes and combinations thereof selected from the panel) may be measured using this method.

The instruments were operated in negative MRM mode. Ionspray voltage was set at −3.5 kV, source temperature at 500° C., curtain gas (e.g., nitrogen) at 20 psi, and nebulizer and desolvation gas (e.g., nitrogen) flow rates at 60 psi, collisionally activated dissociation (CAD) gas (e.g., nitrogen) at medium.

In one example, the eluent from the chromatography column described in Example 1, Chromatography Method 3, was directly and automatically introduced into the electrospray source of a mass spectrometer. Exemplary ions that were generated for the quantitation of xylose, raffinose, and stachyose are listed in Table 7.

TABLE 7
Parent and Daughter Ion Mass-to-Charge Ratios (m/z) of
Analytes from Chromatography Method 3 and MS Method 3

| | | | Approximate |
|---|---|---|---|
| | Parent Ion | Daughter Ions | Retention |
| Analyte | (m/z, ±0.5) | (m/z, ±0.5) | Time (min) |
| xylose | 149.1 | 43.1; 59.1; 71.1; 89.1; 131.1 | 1.5 |
| Xylose-¹³C₅ | 154.1 | 61.1; 92.1; 136.1 | 1.5 |
| raffinose | 503.4 | 323.1; 281.1; 221.1; | 3.8 |
| | | 179.1; 161.1; 119.1; | |
| Raffinose-d₉ | 512.4 | 224.1; 183.1; 164.1 | 3.8 |
| stachyose | 666.6 | 485.3; 383.3; 341.3; | 5.2 |
| | | 221.1; 179.1; | |
| Stachyose-d₇ | 673.6 | 386.1; 345.1; 182.1 | 5.2 |

### Example 3: Quantitative Measurement of Analytes in Solid Experimental Samples

Fecal samples were lyophilized overnight until dry. The dried sample was homogenized, and approximately 20 mg of sample was weighed into a 1.5 mL tube; the exact weight was recorded. In addition to the Experimental fecal samples, Combined Calibration Standards, Blank, Blank-IS and QC samples were prepared for each analytical run. For Calibration Standards, Blank, and Blank-IS samples, 20.0 μL of water was added to 1.5 mL tubes. For QC samples, approximately 20.0 mg of lyophilized QC sample was added to 1.5 mL tubes, and the exact weight was recorded. For Combined Calibration Standards samples, an 80.0 μL volume of Calibration Solution corresponding to the calibration range levels of each of the analyte as determined in Example V. Determination of Calibration Range, was added to corresponding tubes, and an 80.0 μL volume of Ethanol/Acetonitrile/Water (2:1:1) was added to the tubes containing Standard, Blank-IS, QC and Experimental samples. A 20.0 μL volume of the WIS solution was added to tubes containing Calibration Standard, Blank-IS, QC, and Experimental samples, and 20.0 μL of Acetontrile/Water (1:1) was added to the tubes used for the blank samples.

To precipitate proteins and extract analytes, 200 μL of 1% Formic Acid in 70% Methanol was added to samples, and samples were shaken or vortexed for 5 minute and centrifuged for 5 minutes at 4000 rpm. For sample analysis, a 150.0 μL volume of cleared supernatant was transferred into appropriate wells of a fresh 96-well plate. Plates were capped and subject to LC-MS/MS analysis.

Analytes were measured in experimental samples using the LC and MS methods described in Examples 1 and 2. The methods were used to determine the absolute amount of the analytes N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, and stachyose in fecal samples.

The analytes N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), and famotidine were measured using Chromatography Method 1 and MS Method 1 in a single injection with a run time of 7.0 minutes.

The analytes cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, and taurodeoxycholate were measured using Chromatography Method 2 and MS Method 2 in a single injection with a run time of 9.0 minutes.

The analytes xylose, raffinose, and stachyose were measured using Chromatography Method 3 and MS Method 3 in a single injection with a run time of 6.0 minutes.

The measured amounts of the analytes in the samples determined using the described methods are shown in Table 8.

TABLE 8
Results from Representative Fecal Sample

| | Concentration |
|---|---|
| Analyte | (μg/mg) |
| Trimethylamine N-oxide | 0.0646 |
| N-Acetylcadaverine | 46.7 |
| 5-Aminovaleric acid | 156 |
| Cadaverine | 87 |
| Famotidine | 0.000139 |
| gamma-Aminobutyric acid | 21 |
| Serotonin | 3.32 |
| Pipecolic acid | 49.7 |
| Imidazole propionic acid | 0.0172 |
| β-Imidazolelactic acid | 3.79 |
| Cyclo(-His-Pro) | 4.36 |
| Cyclo(-Pro-Thr) | 1.33 |
| Cyclo(-Gly-His) | 1.62 |
| Tryptophan | 118 |
| Hippuric acid | 1.33 |
| 4-hydroxyphenylacetic acid | 15.4 |
| 3-Phenyllactic acid | 41.3 |
| (4-hydroxypheny1)-2-propionic | 5380 |
| acid | |
| (3-Phenylpropiony)lglycine | 0.219 |
| Phenylacetylglycine | 15.4 |
| p-Cresol sulfate | 1.03 |
| Enterodiol | 1.85 |
| Enterolactone | 2.99 |
| Pheno sulfate | 0.0324 |
| p-Cresol glucuronide | 0.0536 |
| Ethylphenyl sulfate | 0.000428 |
| 3-Indoxyl sulfate | 0.0573 |
| Phenylacetic acid | 327 |
| Taurodeoxycholic acid | 4.17 |
| Lactic acid | 360 |
| Deoxycholic acid | 4160 |
| Lithocholic acid | 2510 |
| p-Cresol | 287 |
| Benzoic acid | 5.06 |
| 3-Indolepropionic acid | 4.86 |
| Indole | 111 |
| N-Palmitoyl serinol | 0.0759 |
| Naringenin | 1.2 |
| Daidzein | 0.19 |
| Apigenin | 0.146 |
| Genistein | 0.0565 |
| Equol | 0.0387 |
| Xylose | 61.8 |
| Raffinose | 15.9 |
| Stachyose | 7 |

### Example 4: Quantitative Measurement of Analytes in Liquid Experimental Samples

Serum samples from multiple donors were pooled, and a 50.0 μl aliquot of the pooled sample was added to a well of a microtiter plate. For Blank and Blank-IS samples, 50.0 μL of PBS was added to a well of a microtiter plate. For the Combined Calibration Standards sample, 50.0 μL of Calibration Solutions corresponding to the determined calibration range for each analyte to be measured was added to a well of a microtiter plate. For QC samples, 50.0 μL of the QC sample for the corresponding sample type was added to a well of a microtiter plate. A 20.0 μL volume of the WIS solution was added to the wells containing the Calibration Standard, Blank-IS, QC, and Experimental samples, and 20.0 μL of water was added to the wells containing the blank samples. 40 μL ACN/Water was added to wells with blank samples, and 20 μL ACN/Water was added to wells containing the Blank-IS, QC, and Experimental samples.

To precipitate proteins and extract analytes, 200 μL of methanol was added to all samples, and samples were shaken or vortexed and then centrifuged. A 100 μL volume of cleared supernatant was transferred into a fresh 96-well plate. Plates were capped and subject to LC-MS/MS analysis.

Analytes were measured in experimental samples using the LC and MS methods described in Examples 1 and 2. The methods were used to determine the absolute amount of the analytes catechol sulfate, p-cresol sulfate, ethylphenyl sulfate, indole lactate, indolepropionate, indoxyl sulfate, and trimethylamine-N-oxide (TMAO), in serum samples.

The analytes catechol sulfate, p-cresol sulfate, ethylphenyl sulfate, indole lactate, indolepropionate, and indoxyl sulfate were measured using Chromatography Method 4 and MS Method 2 in a single injection with a run time of 5.50 minutes.

The analyte trimethylamine-N-oxide (TMAO) was measured using Chromatography Method 5 and MS Method 1 in a single injection with a run time of 4.30 minutes.

The measured amounts of the analytes in the representative pooled serum sample determined using the described methods are shown in Table 9.

TABLE 9
Results from Representative Serum Sample

| | Concentration |
|---|---|
| Analyte | (ng/mL) |
| 3-indoxyl sulfate | 152 |
| catechol sulfate | 171 |
| p-cresol sulfate | 72.8 |
| indolelactate | 197 |
| ethylphenyl sulfate | 17.2 |
| 3-indolepropionic acid | 101 |
| trimethylamine N-oxide | 135 |

1. A method for determining the amount of one or a plurality of analytes in a sample by mass spectrometry, wherein the one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof, the method comprising:
a) introducing the sample to an ionization source under conditions suitable to produce one or more ions detectable by mass spectrometry from each of the one or plurality of analytes, wherein the analytes are not derivatized prior to ionization;
b) measuring, by mass spectrometry, the amount of the one or more ions from each of the one or plurality of analytes; and
c) using the measured amount of the one or more ions to determine the amount of each of the one or plurality of analytes in the sample.

2. The method of claim 1, wherein the one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and wherein the amounts of the one or plurality of analytes are determined in a single injection.

3. The method of claim 1, wherein the one or plurality of analytes are selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and wherein the amounts of the one or plurality of analytes are determined in a single injection.

4. The method of claim 1, wherein the one or plurality of analytes are selected from the group consisting of xylose, raffinose, stachyose, N-acetylmuraminate, and N-acetylneuraminate (sialic acid), and wherein the amount(s) of the one or plurality of analytes are determined in a single injection.

5. The method of claim 1, wherein the one or plurality of analytes are selected from the group consisting of catechol sulfate, p-cresol sulfate, ethylphenyl sulfate, indole lactate, indolepropionate, and indoxyl sulfate, and wherein the amount(s) of the one or plurality of analytes are determined in a single injection.

6. The method of claim 1, wherein the sample has been purified by liquid chromatography prior to being introduced to the ionization source.

7. The method of claim 6, wherein the liquid chromatography is selected from the group consisting of high performance liquid chromatography, ultra high performance liquid chromatography, and turbulent flow liquid chromatography.

8. The method of claim 1, wherein the sample has been purified by either high performance liquid chromatography or ultrahigh performance liquid chromatography prior to being introduced to the ionization source.

9. The method of claim 1, wherein the plurality of analytes comprises two or more analytes.

10-14. (canceled)

15. The method of claim 1, wherein the plurality of analytes comprises N-palmitoyl serinol and one or more analytes selected from the group consisting of indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, and 3-indolelactic acid.

16. The method of claim 2, wherein the ionization source is operated in positive ionization mode.

17. The method of claim 3, wherein the ionization source is operated in negative ionization mode.

18. The method of claim 1, wherein
a first one or more analyte(s) of the plurality of analytes is selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and wherein the first one or more analyte(s) of the plurality of analytes are determined in a single injection; and
a second one or more analyte(s) of the plurality of analytes is selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and wherein the second one or more analyte(s) of the plurality of analytes are determined in a single injection.

19. The method of claim 1, wherein
a first one or more analyte(s) of the plurality of analytes is selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and wherein the first one or more analyte(s) of the plurality of analytes are determined in a single injection; and
a second one or more analyte(s) of the plurality of analytes is selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and wherein the second one or more analyte(s) of the plurality of analytes are determined in a single injection; and
a third one or more analyte(s) of the plurality of analytes is selected from the group consisting of xylose, raffinose, stachyose, N-acetylmuraminate, and N-acetylneuraminate (sialic acid), and wherein the third one or more analyte(s) of the plurality of analytes are determined in a single injection.

20. (canceled)

21. The method of claim 20 wherein one of the one or plurality of analytes comprises N-palmitoyl serinol and the one or more ions comprise one or more ions selected from the group consisting of ions with a mass to charge ratio of 330.3±0.5, 312.1±0.5, 239.1±0.5, 149.1±0.5, 139.1±0.5, 92.1±0.5, and 74.1±0.5.

22. The method of claim 1, wherein an internal standard is used to determine the amount of each of the one or plurality of analytes in the sample.

23-25. (canceled)

26. A kit comprising one or more isotopically labeled analogs as internal standards for each of one or a plurality of analytes selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, xylose, raffinose, stachyose, diaminopimelate, trimethylamine (TMA), 3-phenylpropionate, 4-ethylphenol, 4-hydroxyphenyllactate, cinnamate, cinnamoylglycine, phenol glucuronide, urolithin A, N-acetylmuraminate, N-acetylneuraminate (sialic acid), catechol sulfate, 3-indolelactic acid, and combinations thereof, and packaging material and instructions for using the kit.

27. The kit of claim 26, wherein the one or plurality of analytes are selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, trimethylamine (TMA), and combinations thereof.

28. The kit of claim 26, wherein the one or plurality of analytes are selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, urolithin A, and combinations thereof.

29-35. (canceled)

36. The method of claim 1, wherein
a first one or more analyte(s) of the plurality of analytes is selected from the group consisting of N-palmitoyl serinol, indolepropionate, indole, tryptophan, 5-aminovalerate, pipecolate, N-acetyl-cadaverine, cadaverine, trimethylamine-N-oxide (TMAO), gamma-aminobutyric acid (GABA), serotonin, imidazole propionate, imidazole lactate, cyclo(-His-Pro), cyclo(-Pro-Thr), cyclo(-Gly-His), famotidine, diaminopimelate, and trimethylamine (TMA), and wherein the first one or more analyte(s) of the plurality of analytes are determined in a single injection; and
a second one or more analyte(s) of the plurality of analytes is selected from the group consisting of cresol, 3-indoxyl sulfate, 4-hydroxyphenylacetate, 2-(4-hydroxyphenyl)propionate, benzoate, phenylacetic acid, phenyllactate, hippurate, lactate, phenylpropionylglycine, phenylacetylglycine, ethylphenyl sulfate, phenol sulfate, p-cresol sulfate, p-cresol glucuronide, enterodiol, enterolactone, equol, daidzein, apigenin, naringenin, genistein, deoxycholate, lithocholate, taurodeoxycholate, 3-phenylpropionate, 4-ethylphenol, 4-hydroxyphenyllactate, cinnamate, cinnamoylglycine, phenol glucuronide, and urolithin A, and wherein the second one or more analyte(s) of the plurality of analytes are determined in a single injection;
a third one or more analyte(s) of the plurality of analytes is selected from the group consisting of xylose, raffinose, stachyose, N-acetylmuraminate, and N-acetylneuraminate (sialic acid), and wherein the third one or more analyte(s) of the plurality of analytes are determined in a single injection; and
a fourth one or more analyte(s) of the plurality of analytes is selected from the group consisting of catechol sulfate, p-cresol sulfate, ethylphenyl sulfate, indole lactate, indolepropionate, and indoxyl sulfate, and wherein the fourth one or more analyte(s) of the plurality of analytes are determined in a single injection.

37-38. (canceled)
